(19) United States
(12) Patent Application Publication
(10) Pub. No.: US 2010/0105041 A1
(43) Pub. Date: Apr. 29, 2010

(54) REAGENTS AND METHODS FOR DETECTING CYP2D6 POLYMORPHISMS

(75) Inventors: Lailing Ku, Pleasanton, CA (US); Charlene Bush-Donovan, Livermore, CA (US); Lu-ping Shen, San Leandro, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS HEALTHCARE DIAGNOSTICS INC., Tarrytown, NY (US)

(21) Appl. No.: 12/518,697

(22) PCT Filed: Dec. 14, 2007
(86) PCT No.: PCT/US07/87513

§ 371 Date: Dec. 1, 2009

Related U.S. Application Data
(60) Provisional application No. 60/874,840, filed on Dec. 14, 2006.

(51) Int. Cl. C12Q 1/68; C07H 21/04

(52) U.S. Cl. 435/6; 536/23.1; 536/24.33; 536/24.3

(57) ABSTRACT

The present invention relates to oligonucleotide sequences for amplification primers and detection probes and their use in nucleic acid amplification methods for the specific detection of clinically relevant CYP2D6 polymorphisms, in particular CYP2D6 polymorphisms associated with adverse drug response. The oligonucleotide sequences are also provided assembled as kits that can be used to predict how an individual will respond to drugs or other xenobiotic compounds that are metabolized, at least in part, by CYP2D6.

### CROSS-REFERENCE TO RELATED APPLICATIONS

This application claims priority to U.S. provisional application No. 60/874,840 filed Dec. 14, 2006, the entire contents of which are hereby incorporated by reference.

### BACKGROUND OF THE INVENTION

It is well recognized that individuals exhibit considerable variability with respect to their response to pharmaceutical agents and other chemicals. Some drugs work well in some patient populations and not as well in others. Some patients experience undesirable or even toxic side effects at drug doses that would be considered appropriate for use in a typical individual, while in other cases a higher than usual dose is required for efficacy. This variability in drug response poses a significant challenge both in terms of selecting appropriate therapeutic agents and doses for the individual patient and in terms of predicting dosing, safety, and efficacy for newly developed drugs. It has been estimated that adverse drug reactions attributed to drugs that were “properly prescribed and administered” result in over 100,000 deaths annually in the United States alone (K. Lazarou et al., JAMA, 1998, 279: 1200-1205). Individual variability in drug response may well be at least in part responsible for a significant fraction of these poor outcomes as well as for the therapeutic failures that are frequently encountered in a wide range of diseases.

One of the major determinants of inter-individual variability in drug response is the existence, among individuals, of differences in genes that encode enzymes responsible for various aspects of drug metabolism. The science of pharmacogenetics encompasses the identification and analysis of differences in genetic makeup that influence response to drug treatment. Once a correlation between genotype and drug response has been established, information about an individual patient's genotype can be used to guide the choice of appropriate therapeutic agents and/or the selection of an appropriate dose of an agent for that patient. For example, if a patient is recognized as having a genotype associated with reduced metabolism of a particular therapeutic agent relative to metabolism of that agent in most individuals, that agent could be avoided or the dose reduced, or the patient could be closely monitored for toxicity.

Enzymes involved in the bio-transformation of drugs are also responsible for bio-transformation of other xenobiotics, including chemicals encountered in the environment or workplace that have been linked to disease. Thus, understanding inter-individual differences in the metabolism of these compounds would help in identifying persons who may be at particular risk so that appropriate measures could be taken to minimize such risk. Identification and analysis of genetic polymorphisms that are associated with differences in the metabolism of drugs and other xenobiotics is thus of great interest, and considerable progress in this area has been made.

Enzymes of the cytochrome P450 family play a major role in the bio-transformation of drugs and other xenobiotics as well as a variety of endogenous substances. These enzymes are predominantly found in the liver and are responsible for metabolizing more than 50% of all currently marketed drugs. Polymorphisms at the cytochrome P450 2D6 (CYP2D6) locus are a common cause of pharmacogenetic variability in humans. CYP2D6, also known as debrisoquine 4-hydroxylase, is involved in the metabolism of approximately 30-50% of all therapeutic agents. CYP2D6 metabolizes numerous classes of drugs including anti-arrhythmics, anti-hypertensives, beta-blockers, opioids, anti-psychotics, and anti-depressants as well as a variety of compounds encountered in the environment.

CYP2D6 has a wide range of activity within human populations, with rates of CYP2D6-mediated metabolism varying by a factor of more than 10,000 among individuals as a result of the existence of different CYP2D6 alleles associated with varying levels of CYP2D6 activity. Most individuals are able to metabolize CYP2D6 substrates extensively and are classified as having an extensive metabolizer (EM) phenotype. Individuals who fail to produce functional CYP2D6 exhibit a poor metabolizer (PM) phenotype and typically have two defective CYP2D6 alleles or a whole deletion of the CYP2D6 gene. Individuals with an intermediate metabolizer (IM) phenotype have a rate of metabolism between that of poor and extensive metabolizers as a consequence of partially defective CYP2D6 alleles. Individuals with duplicated or amplified functional CYP2D6 alleles exhibit an ultra-rapid metabolizer (UM) phenotype (I. Johannson et al., Proc. Natl. Acad. Sci. USA, 1993, 90: 11825-11829; R. Lovlie et al., FEBS Letters, 1996, 392: 30-34).

It is evident that a significant potential exists for major differences in response to drug metabolized by CYP2D6. For example, genetic polymorphism in CYP2D6 is responsible for considerable inter-individual variability in the metabolism of the tricyclic anti-depressants nortryptiline, and this variability can have undesirable and even life-threatening consequences. CYP2D6 poor metabolizers can experience severe adverse effects as a result of high nortryptiline concentrations, while ultra-rapid metabolizers may experience a lack of efficacy (L. Bertilsson et al., Br. J. Clin. Pharmacol., 2002, 53: 111-122). Certain drugs are metabolized to an active form by CYP2D6. For example, O-demethylation of codeine into morphine by CYP2D6 is essential for its opioid activity. Codeine is therefore ineffective in individuals lacking at least one functional CYP2D6 allele (S. H. Sindrup et al., Pharmacogenetics, 1995, 5: 335-346). On the other hand, ultra-rapid metabolism of codeine can lead to toxicity in patients in which other pathways of codeine elimination are compromised (Y. Gasche et al., N. Engl. J. Med., 2004, 351: 2827-2831). Variability in CYP2D6 activity can also lead to drug interactions and increased susceptibility to certain diseases, which may at least in part be mediated by compounds that are normally metabolized by CYP2D6.

A variety of methods have been developed to assess CYP2D6 activity. Many of these methods involve administration of a test compound (e.g., debrisoquine, sparteine, or dextromethorphan) to a subject and measurement of its metabolism by CYP2D6-mediated pathways. The discovery of genetic polymorphisms responsible for inter-individual differences in CYP2D6 has allowed the development of assays based on detecting variations in the sequence of the CYP2D6 gene and/or the presence of duplication or amplification thereof. Examples of such assays are described, for example, in U.S. Pat. Nos. 5,648,482; 5,981,174; and 6,183,963. However, there remains a need for improved methods of detecting polymorphisms in CYP2D6 and for genotyping individuals with respect to their CYP2D6 alleles.

### SUMMARY OF THE INVENTION

The present invention is directed to systems for the rapid, reliable, and convenient detection of multiple CYP2D6 polymorphisms of clinical significance. In particular, the present invention provides reagents and methods for the detection of polymorphisms of CYP2D6 associated with adverse drug response. More specifically, the present invention provides CYP2D6-specific oligonucleotide sequences for amplification primers and detection probes that can be used to detect multiple CYP2D6 single nucleotide polymorphisms (SNPs) as well as CYP2D6 gene duplication and CYP2D6 gene deletion. In certain embodiments, the inventive oligonucleotide sequences are useful for the detection of CYP2D6*2A, CYP2D6*12, CYP2D6*4, CYP2D6*10, CYP2D6*11, CYP2D6*17, CYP2D6*6, CYP2D6*8, CYP2D6*3, CYP2D6*9, CYP2D6*2, CYP2D6*7, CYP2D6*5 (gene deletion), and CYP2D6 gene duplication

In one aspect, the present invention provides isolated oligonucleotides comprising a nucleic acid sequence selected from the group consisting of SEQ ID NOs. 1-88, complementary sequence thereof, active fragments thereof, and combinations thereof. These isolated oligonucleotides can be used for amplifying a portion of a CYP2D6 gene or a portion of genomic DNA indicative of CYP2D6 deletion or duplication and/or for detecting a CYP2D6 gene polymorphism.

In another aspect, the present invention provides a primer pair for amplifying a portion of a CYP2D6 gene or portion of genomic DNA comprising a CYP2D6 deletion or duplication by PCR, wherein the primer pair is selected from the group consisting of: (a) Primer Pair 1 comprising primers having nucleotide sequences set forth in SEQ ID NOs. 1 and 2; (b) Primer Pair 2 comprising primers having nucleotide sequences set forth in SEQ ID NOs. 3 and 4; (c) Primer Pair 3 comprising primers having nucleotide sequences set forth in SEQ ID NOs. 5 and 7; (d) Primer Pair 4 comprising primers having nucleotide sequences set forth in SEQ ID NOs. 6 and 7; (e) Primer Pair 5 comprising primers having nucleotide sequences set forth in SEQ ID NOs. 6 and 8; and (f) Primer Pair 6 comprising primers having nucleotide sequences set forth in SEQ ID NOs. 9 and 10.

In still another aspect, the present invention provides a pair of allele-specific extension probes that can distinguish between CYP2D6 alleles that differ at a polymorphic position when used in a primer extension assay, wherein one of said extension probes is complementary to a wild-type CYP2D6 allele at the polymorphic position and the other of said extension probes is complementary to a mutant CYP2D6 allele at the polymorphic position, wherein said polymorphic position is selected from the group consisting of: −15841 100, 124, 883, 1023, 1707, 1758, 1846, 2549, 2613-2615, 2850 and 2935.

In certain embodiments, the pair of allele-specific extension probes is selected from the group consisting of: a probe pair comprising a wild-type probe and a mutant probe having sequences as set forth in SEQ ID NOs 11 and 12; a probe pair comprising a wild-type probe and a mutant probe having sequences selected from the group consisting of SEQ ID NOs. 13-24; a probe pair comprising a wild-type probe and a mutant probe having sequences selected from the group consisting of SEQ ID NOs. 25-32; a probe pair comprising a wild-type probe and a mutant probe having sequences selected from the group consisting of SEQ ID NOs. 33-36; a probe pair comprising a wild-type probe and a mutant probe having sequences selected from the group consisting of SEQ ID NOs. 37-44; a probe pair comprising a wild-type probe and a mutant probe having sequences selected from the group consisting of SEQ ID NOs. 45-52; a probe pair comprising a wild-type probe and a mutant probe having sequences selected from the group consisting of SEQ ID NOs. 53-56; a probe pair comprising a wild-type probe and a mutant probe having sequences selected from the group consisting of SEQ ID NOs. 57-65; a probe pair comprising a wild-type probe and a mutant probe having sequences as set forth in SEQ ID NOs 66 and 67; a probe pair comprising a wild-type probe and a mutant probe having sequences selected from the group consisting of SEQ ID NOs. 68-75; a probe pair comprising a wild-type probe and a mutant probe having sequences selected from the group consisting of SEQ ID NOs. 76-81; and a probe pair comprising a wild-type probe and a mutant probe having sequences as set forth in SEQ ID NOs. 82 and 83.

In yet another aspect, the present invention provides a kit comprising a collection of primer pairs, wherein said primer pairs are suitable for use in a single-plex or multiplex PCR reaction that comprises human genomic DNA, said collection comprising: (a) a primer pair that, when used in the PCR reaction, generates an amplification product that encompasses nucleotides 5173 to 8953 of the CYP2D6 gene (Accession NG 003180); (b) a primer pair that, when used in the PCR reaction, generates an amplification product that encompasses nucleotides 2922 to 8953 of the CYP2D6 gene (Accession M_{—}33388); (c) a primer pair that, when used in the PCR reaction, generates an amplification product only if the genomic DNA contains a CYP2D6 deletion; and (d) a primer pair that, when used in the PCR reaction, generates and amplification product only if the genomic DNA contains a CYP2D6 duplication. In certain preferred embodiments, the primer pairs do not significantly amplify CYP2D7 and/or CYP2D8 sequences present in the PCR reaction.

In certain embodiments, the kit comprises the following primer pair: (a) a primer pair comprising primers having nucleotide sequences set forth in SEQ ID NOs. 1 and 2; (b) a primer pair comprising primers having nucleotide sequences set forth in SEQ ID NOs. 3 and 4; (c) at least one primer pair selected from the group consisting of: (i) primer pair 3 comprising primers having s nucleotide sequences set forth in SEQ ID NOs. 5 and 7; (ii) primer pair 4 comprising primers having nucleotide sequences set forth in SEQ ID NOs. 6 and 7; and (iii) primer pair 5 comprising primers having nucleotide sequences set forth in SEQ ID NOs. 6 and 8; and (d) a primer pair comprising primers having nucleotide sequences set forth in SEQ ID NOs. 9 and 10.

In another aspect, the present invention provides a primer/probe set for detecting a CYP2D6 polymorphism, wherein the primer/probe set is selected from the group consisting of: (a) a primer pair comprising primers having sequences set forth in SEQ ID NOs. 1 and 2; and at least one probe pair selected from the group consisting of: a probe pair comprising a wild-type probe and a mutant probe having sequences as set forth in SEQ ID NOs 11 and 12; a probe pair comprising a wild-type probe and a mutant probe having sequences selected from the group consisting of SEQ ID NOs. 13-24; a probe pair comprising a wild-type probe and a mutant probe having sequences selected from the group consisting of SEQ ID NOs. 25-32; a probe pair comprising a wild-type probe and a mutant probe having sequences selected from the group consisting of SEQ ID NOs. 33-36; and a probe pair comprising a wild-type probe and a mutant probe having sequences selected from the group consisting of SEQ ID NOs. 37-44; (b) a primer pair comprising primers having sequences set forth in SEQ ID NOs. 3 and 4; and at least one probe pair selected from the group consisting of: a probe pair comprising a wild-type probe and a mutant probe having sequences selected from the group consisting of SEQ ID NOs. 45-52; a probe pair comprising a wild-type probe and a mutant probe having sequences selected from the group consisting of SEQ ID NOs. 53-56; a probe pair comprising a wild-type probe and a mutant probe having sequences selected from the group consisting of SEQ ID NOs. 57-65; a probe pair comprising a wild-type probe and a mutant probe having sequences as set forth in SEQ ID NOs 66 and 67; a probe pair comprising a wild-type probe and a mutant probe having sequences selected from the group consisting of SEQ ID NOs. 68-75; a probe pair comprising a wild-type probe and a mutant probe having sequences selected from the group consisting of SEQ ID NOs. 76-81; and a probe pair comprising a wild-type probe and a mutant probe having sequences as set forth in SEQ ID NOs. 82 and 83; (c) a primer pair comprising primers having sequences set forth in SEQ ID NOs. 5 and 7; and at least one probe selected from the group consisting of probe having sequence set forth in SEQ ID NO. 84 and probe having sequence set forth in SEQ ID NO. 85; (d) a primer pair comprising primers having sequences set forth in SEQ ID NOs. 6 and 7; and at least one probe selected from the group consisting of probe having sequence set forth in SEQ ID NO. 84 and probe having sequence set forth in SEQ ID NO. 85; (e) a primer pair comprising primers having sequences set forth in SEQ ID NOs. 6 and 8; and at least one probe selected from the group consisting of probe having sequence set forth in SEQ ID NO. 84 and probe having sequence set forth in SEQ ID NO. 85; (f) a primer pair comprising primers having sequences set forth in SEQ ID NOs. 9 and 10; and at least one probe selected from the group consisting of probe having sequence set forth in SEQ ID NO. 86, probe having sequence set forth in SEQ ID NO. 87 and probe having sequence set forth in SEQ ID NO. 88.

In another aspect, the present invention provides a kit comprising a collection of primer pairs, wherein said primer pairs are suitable for use in a single or multiplex PCR reaction that comprises human genomic DNA, said collection comprising: (a) a primer pair that, when used in the PCR reaction, generates an amplification product that encompasses nucleotides 5173 to 8953 of the CYP2D6 gene (Accession NG 003180); (b) a primer pair that, when used in the PCR reaction, generates an amplification product that encompasses nucleotides 2922 to 4730 of the CYP2D6 gene Accession M_{—}33388); (c) a primer pair that, when used in the PCR reaction, generates an amplification product only if the genomic DNA contains a CYP2D6 deletion; and (d) a primer pair that, when used in the PCR reaction, generates and amplification product only if the genomic DNA contains a CYP2D6 duplication. In certain preferred embodiments, the primer pairs do not significantly amplify CYP2D7 and/or CYP2D8 sequences present in the PCR reaction.

In certain embodiments, the kit comprises the following primer pairs: (a) primer pair 1 comprising primers having nucleotide sequences set forth in SEQ ID NOs. 1 and 2; (b) primer pair 2 comprising primers having nucleotide sequences set forth in SEQ ID NOs. 3 and 4; (c) at least one primer pair selected from the group consisting of: (i) primer pair 3 comprising primers having sequences set forth in SEQ ID NOs. 5 and 7; (ii) primer pair 4 comprising primers having sequences set forth in SEQ ID NOs. 6 and 7; and (iii) primer pair 5 comprising primers having sequences set forth in SEQ ID NOs. 6 and 8; and (d) primer pair 1 comprising primers having nucleotide sequences set forth in SEQ ID NOs. 9 and 10.

In certain embodiments, the kit further comprises a collection of probes comprising: (a′) at least one probe pair that can be used in an ASPE reaction to detect a SNP that resides within the amplification product generated by the primer pair set forth in (a); (b′) at least one probe pair that can be used in an ASPE reaction to detect a SNP that resides within the amplification product generated by the primer pair set forth in (b); (c′) at least one probe that hybridizes to the amplification product generated by the primer pair set forth in (c); and (d′) at least one probe that hybridizes to the amplification product generated by the primer pair set forth in (d).

In some embodiments, the kit comprises a collection of probes comprising: (a′) at least one probe pair selected from the group consisting of: a probe pair comprising a wild-type probe and a mutant probe having sequences as set forth in SEQ ID NOs 11 and 12; a probe pair comprising a wild-type probe and a mutant probe having sequences selected from the group consisting of SEQ ID NOs. 13-24; a probe pair comprising a wild-type probe and a mutant probe having sequences selected from the group consisting of SEQ ID NOs. 25-32; a probe pair comprising a wild-type probe and a mutant probe having sequences selected from the group consisting of SEQ ID NOs. 33-36; and a probe pair comprising a wild-type probe and a mutant probe having sequences selected from the group consisting of SEQ ID NOs. 37-44; (b′) at least one probe pair selected from the group consisting of: a probe pair comprising a wild-type probe and a mutant probe having sequences selected from the group consisting of SEQ ID NOs. 45-52; a probe pair comprising a wild-type probe and a mutant probe having sequences selected from the group consisting of SEQ ID NOs. 53-56; a probe pair comprising a wild-type probe and a mutant probe having sequences selected from the group consisting of SEQ ID NOs. 57-65; a probe pair comprising a wild-type probe and a mutant probe having sequences as set forth in SEQ ID NOs 66 and 67; a probe pair comprising a wild-type probe and a mutant probe having sequences selected from the group consisting of SEQ ID NOs. 68-75; a probe pair comprising a wild-type probe and a mutant probe having sequences selected from the group consisting of SEQ ID NOs. 76-81; and a probe pair comprising a wild-type probe and a mutant probe having sequences as set forth in SEQ ID NOs. 82 and 83; (c′) at least one probe selected from the group consisting of: a probe having sequence set forth in SEQ ID NO. 84 and a probe having sequence set forth in SEQ ID NO. 85; and (d′) at least one probe selected from the group consisting of: a probe having sequence set forth in SEQ ID NO. 86, a probe having sequence set forth in SEQ ID NO. 87 and a probe having sequence set forth in SEQ ID NO. 88.

The probes may be attached to a solid support. For example, the probes may be attached to microparticles, or to an array. In certain embodiments, the kit further comprises reagent for performing a Luminex assay.

In another aspect, the present invention provides a CYP2D6 amplification product generated by a PCR reaction containing a human genomic DNA and at least one primer pair as disclosed herein. The present invention also provides a collection of CYP2D6-related amplification products, wherein said collection comprises at least two amplification products generated by a PCR reaction, said PCR reaction containing human genomic DNA and at least two primer pairs disclosed herein. In certain embodiments, human genomic allele comprises a CYP2D6 allele selected from the group consisting of CYP2D6*2A, CYP2D6*12, CYP2D6*4, CYP2D6*10, CYP2D6*11, CYP2D6*17, CYP2D6*6, CYP2D6*8, CYP2D6*3, CYP2D6*9, CYP2D6*2, CYP2D6*7, CYP2D6*5 (gene deletion), and CYP2D6 gene duplication.

In still another aspect, the present invention provides a method for determining which of a plurality of CYP2D6 polymorphic variants is present in an individual The method comprises steps of: (a) contacting a sample containing nucleic acid derived from the individual with at least one allele-specific extension probe, wherein said extension probe is complementary to genomic DNA comprising a CYP2D6 gene sequence and terminates at its 3′ end at a polymorphic position in the CYP2D6 gene sequence, so that the probe hybridizes to a CYP2D6 polymorphic variant that contains a nucleotide complementary to the 3′ terminal nucleotide of the probe; (b) subjecting a nucleic acid hybrid formed by hybridization of the probe and nucleic acid comprising a CYP2D6 gene sequence to conditions suitable for primer extension; and (c) detecting extension of the allele-specific primer, wherein extension of the allele-specific primer is indicative of the presence of one of a plurality of CYP2D6 polymorphic variants in the individual.

In certain embodiments, the plurality of CYP2D6 polymorphic variants is selected from the group consisting of: −1584 C>G, 100 C>T, 124 G>A, 883 G>C, 1023 C>T, 1707 T>del, 1758 G>T, 1846 G>A, 2549 A>del, 2613-2615 del AGA, 2850 C>T, and 2935 A>C.

In certain embodiments, the extension probe has a sequence selected from the group consisting of SEQ ID NOs. 11-83.

In certain embodiments, the step of contacting comprises contacting the nucleic acid with a plurality of allele-specific probes, said plurality of allele-specific extension probes comprising at least one pair of extension probes comprising a first extension probe comprising a 3′ portion that hybridizes to a target region of genomic DNA comprising a CYP2D6 gene sequence immediately adjacent to a polymorphic position and that has a 3′-terminal nucleotide that is complementary to a non-mutated/wild-type base at said polymorphic position, and a second extension probe comprising a 3′ portion that hybridizes to a target region of genomic DNA comprising a CYP2D6 gene sequence immediately adjacent to the polymorphic position and that has a 3′-terminal nucleotide that is complementary to a mutated/mutant base at said polymorphic position.

In such methods, the sample may comprise DNA obtained by amplification, for example, said amplification is/was performed using a plurality of primers having sequences selected from the group consisting of SEQ ID NOs. 1-10.

In certain embodiments, the detecting step comprises determining which of at least two polymorphic variants exists at a polymorphic site.

In some embodiments, the methods further comprise a step of selecting a therapeutic regimen for the individual, wherein the therapeutic regimen is selected, at least in part, on the basis of the presence of one or more of the plurality of CYP2D6 polymorphic variants in the individual.

These and other objects, advantages and features of the present invention will become apparent to those of ordinary skill in the art having read the following detailed description of the preferred embodiments.

### BRIEF DESCRIPTION OF THE DRAWING

Table 1 shows examples of inventive specific amplification primer sequences that can be used in either singlex amplification or multiplex amplification of the CYP2D6 gene.

Table 2 shows examples of inventive detection probe sequences that can be used in a multiplex detection of the CYP2D6 gene.

FIG. 1 presents agarose gel electrophoretic profiles of long PCR amplification products that include 2D6 gene SNP sites.

FIG. 2 presents results of P450 2D6 assay determination based on Luminex data analysis. As shown on this figure, the individual tested is heterozygous for 1023 C>T and a homozygous mutant for 2850 C>T, and wild-type for other polymorphisms.

### DEFINITIONS

Throughout the specification, several terms are employed that are defined in the following paragraphs.

The term “gene”, as used herein, has its art understood meaning, and refers to a part of the genome specifying a macromolecular product, be it a functional RNA molecule or a protein, and may include regulatory sequences (e.g., promoters, enhancers, etc) and/or intro sequences preceding (5′ non-coding sequences) and following (3′ non-coding sequences) the coding sequences. For example, as used herein, the CYP2D6 gene includes the CYP2D6 promoter region, as well as non-coding nucleic acid sequence that is present in the CYP2D6 transcript (e.g., 5′ and/or 3′ unstranslated regions).

A “gene product” or “expression product” is an RNA transcribed from the gene (e.g., either pre- or post-processing) and/or a polypeptide encoded by an RNA transcribed from the gene (e.g., either pre- or post-modification). An RNA transcribed from a gene or polynucleotide is said to be encoded by the gene or polynucleotide. Similarly, a polypeptide generated by translation of a messenger RNA is said to be encoded by that messenger RNA, and is also said to be encoded by the gene from which the messenger RNA is transcribed.

As used herein, the term “wild-type” refers to a gene, gene portion or gene product that has the characteristics of that gene, gene portion or gene product when isolated from a naturally-occurring source. A wild-type gene has the sequence that is the most frequently observed in a population and is thus arbitrarily designated as the “normal” or “wild-type” sequence.

The terms “allele” and “allelic variant” are used herein interchangeably. They refer to alternative forms of a gene or a gene portion. Alleles occupy the same locus or portion on homologous chromosomes. When an individual has two identical alleles of a gene, the individual is said to be homozygous for the gene or allele. When an individual has two different alleles of a gene, the individual is said to be heterozygous for the gene. Alleles of a specific gene can differ from each other in a single nucleotide or a plurality of nucleotides, and can include substitutions, deletions and/or insertions of nucleotides with respect to each other. An allele of a gene can also be a form of a gene containing a mutation. While the terms “allele” and “allelic variant” have traditionally been applied in the context of genes, which can include a plurality of polymorphic sites, the term may also be applied to any form of a genomic DNA sequence, which may or may not fall within a gene. Thus each polymorphic variant of a polymorphic site can be considered as an allele of that site. The term “allele frequency” refers to the frequency at which a particular polymorphic variant, or allele, occurs in a population being tested (e.g., between cases and controls in an association study).

The term “polymorphism” refers to the occurrence of two or more alternative genomic DNA sequences or alleles that exist and are inherited within a population. Either of the sequences themselves, or the site at which they occur, may also be referred to as a polymorphism. If a polymorphism is located within a portion of the genome that is transcribed into RNA, the collective RNA of that population will also contain a polymorphism at that position. A “single nucleotide polymorphism or SNP” is a polymorphism that exists at a single nucleotide position. A “polymorphic site”, “polymorphic position” or “polymorphic locus” is a location at which differences in genomic DNA exist among members of a population. While in general the polymorphic sites of interest in the context of the present invention are single nucleotides, the term is not limited to sites that are only one nucleotide in length. A “polymorphic region” is a region of genomic DNA that includes one or more polymorphic sites.

The term “polymorphic variant” refers to any of the alternate sequences that may exist at a polymorphic site among members of a population. For purpose of the present invention, the population may be the population of the world, or a subset thereof. For the methods described herein, it will typically be of interest to determine which polymorphic variant(s) (as among multiple polymorphic variants that exist within a population) is/are present in an individual.

As used herein, the term “genotype” refers to the identity of an allelic variant at a particular polymorphic position in an individual. It will be appreciated that an individual's genome will contain two allelic variants for each polymorphic position (located on homologous chromosomes). The allelic variants can be the same or different. A genotype can include the identity of either or both alleles. A genotype can include the identities of allelic variants at multiple different polymorphic positions, which may or may not be located within a single gene. A genotype can also refer to the identity of an allele of a gene at a particular gene locus in an individual and can include the identity of either or both alleles. The identity of the allele of a gene may include the identity of the polymorphic variants that exist at multiple polymorphic sites within the gene. The identity of an allelic variant or an allele of a gene refers to the sequence of the allelic variant or allele of a gene (e.g., the identity of the nucleotide present at a polymorphic position or the identities of nucleotides present at each of the polymorphic positions in a gene). It will be appreciated that the identity need not be provided in terms of the sequence itself. For example, it is typical to assign identifiers such as +, −, A, a, B, b, etc to different allelic variants or alleles for descriptive purposes. Any suitable identifier can be used. “Genotyping” an individual refers to providing the genotype of the individual with respect to one or more allelic variants or alleles.

The terms “individual” and “subject” are used herein interchangeably. They refer to a human being. The terms do not denote a particular age, and thus encompass adults, children, newborns, as well as fetuses.

As used herein, a “sample” obtained from an individual may include, but is not limited to, any or all of the following: a cell or cells, a portion of tissue, blood, serum, ascites, urine, saliva, amniotic fluid, cerebrospinal fluid, and other body fluids, secretion, or excretions. The sample may be a tissue sample obtained, for example, from skin, muscle, buccal or conjunctival mucosa, placenta, gastrointestinal tract or other organs. A sample of DNA from fetal or embryonic cells or tissue can be obtained by appropriate methods, such as by amniocentesis or chorionic villus sampling. Samples may also include sections of tissues such as frozen sections. The term “sample” also includes any material derived by isolating, purifying, and/or processing a sample as previously defined. Derived materials include, but are not limited to, cells (or their progeny) isolated from the sample, cell components, nucleic acids or proteins extracted from the sample or obtained by subjecting the sample to techniques such as amplification or reverse transcription of mRNA, etc. Processing of the sample may involve one or more of: filtration, distillation, centrifugation, extraction, concentration, dilution, purification, inactivation of interfering components, addition of reagents, and the like.

The terms “genomic DNA” and “genomic nucleic acid” are used herein interchangeably. They refer to nucleic acid from the nucleus of one or more cells, and include nucleic acid derived from (e.g., isolated from, cloned from) genomic DNA. The terms “sample of genomic DNA” and “sample of genomic nucleic acid” are used herein interchangeably and refer to a sample comprising DNA or nucleic acid representative of genomic DNA isolated from a natural source and in a form suitable for hybridization to another nucleic acid (e.g., as a soluble aqueous solution). Samples of genomic DNA to be used in the practice of the present invention generally include a plurality of nucleic acid segments (or fragments) which together may cover a substantially complete genome or a portion of the genome comprising the CYP2D6 gene or a genomic sequence indicative of CYP2D6 deletion or duplication. A sample of genomic DNA may be isolated, extracted or derived from solid tissues, body fluids, skeletal tissues, or individual cells. A sample of genomic DNA can be isolated, extracted or derived from fetal or embryonic cells or tissues obtained by appropriate methods, such as amniocentesis or chronic villus sampling.

The terms “nucleic acid”, “nucleic acid molecule”, and “polynucleotide” are used herein interchangeably. They refer to linear polymers of nucleotide monomers or analogs thereof, such as deoxyribonucleic acid (DNA) and ribonucleic acid (RNA). Unless otherwise stated, the terms encompass nucleic acid-like structures with synthetic backbones, as well as amplification products.

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR) and ligase chain reaction (LCR). In a typical PCR amplification reaction, a nucleic acid sequence of interest is often amplified at least fifty thousand fold in amount over its amount in the starting sample. A “copy” or “amplicon” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification.

The term “oligonucleotide”, as used herein, refers to a short string of nucleotides or analogs thereof. These short stretches of nucleic acid sequences may be obtained by a number of methods including, for example, chemical synthesis, restriction enzyme digestion or PCR. As will be appreciated by one skilled in the art, the length of an oligonucleotide (i.e., the number of nucleotides) can vary widely, often depending on its intended function or use. Generally, oligonucleotides comprise between about 5 and about 150 nucleotides, usually between about 10 and about 100 nucleotides, and more usually between about 15 and about 50 nucleotides. Throughout the specification, whenever an oligonucleotide is represented by a sequence of letters (chosen from the four base letters: A, C, G, and T, which denote adenosine, cytidine, guanosine, and thymidine, respectively), the nucleotides are presented in the 5′→3′ order from the left to the right.

The term “3′” refers to a region or position in a polynucleotide or oligonucleotide 3′ (i.e., downstream) from another region or position in the same polynucleotide or oligonucleotide. The term “5′” refers to a region or position in a polynucleotide or oligonucleotide 5′ (i.e., upstream) from another region or position in the same polynucleotide or oligonucleotide. The terms “3′ end” and “3′ terminus”, as used herein in reference to a nucleic acid molecule, refer to the end of the nucleic acid which contains a free hydroxyl group attached to the 3′ carbon of the terminal pentose sugar. The term “5′ end” and “5′ terminus”, as used herein in reference to a nucleic acid molecule, refers to the end of the nucleic acid molecule which contains a free hydroxyl or phosphate group attached to the 5′ carbon of the terminal pentose sugar.

The term “isolated”, as used herein in reference to an oligonucleotide, means an oligonucleotide, which by virtue of its origin or manipulation, is separated from at least some of the components with which it is naturally associated or with which it is associated when initially obtained. By “isolated”, it is alternatively or additionally meant that the oligonucleotide of interest is produced or synthesized by the hand of man.

The terms “target nucleic acid” and “target sequence” are used herein interchangeably. They refer to a nucleic acid sequence, the presence or absence of which is desired to be determined/detected. The target sequence may be single-stranded or double-stranded. If double-stranded, the target sequence may be denatured to a single-stranded form prior to its detection. This denaturation is typically performed using heat, but may alternatively be carried out using alkali, followed by neutralization. In the context of the present invention, a target sequence comprises at least one single nucleotide polymorphic site. Preferably, target sequences comprise nucleic acid sequences to which primers can hybridize, and/or probe-hybridization sequences with which probes can form stable hybrids under desired conditions.

The term “hybridization”, as used herein, refers to the formation of complexes (also called duplexes or hybrids) between nucleotide sequences which are sufficiently complementary to form complexes via Watson-Crick base pairing or non-canonical base pairing. It will be appreciated that hybridizing sequences need not have perfect complementarity to provide stable hybrids. In many situations, stable hybrids will form where fewer than about 10% of the bases are mismatches. Accordingly, as used herein, the term “complementary” refers to a nucleic acid molecule that forms a stable duplex with its complement under assay conditions, generally where there is about 90% or greater homology. Those skilled in the art understand how to estimate and adjust the stringency of hybridization conditions such that sequences that have at least a desired level of complementarity will stably hybridize, while those having lower complementarity will not. For examples of hybridization conditions and parameters, see, for example, J. Sambrook et al., “Molecular Cloning: A Laboratory Manual”, 1989, Second Edition, Cold Spring Harbor Press: Plainview, N.Y.; F. M. Ausubel, “Current Protocols in Molecular Biology”, 1994, John Wiley & Sons: Secaucus, N.J. Complementarity between two nucleic acid molecules is said to be “complete”, “total” or “perfect” if all the nucleic acids' bases are matched, and is said to be “partial” otherwise.

The terms “probes” and “primers”, as used herein, typically refer to oligonucleotides that hybridize in a sequence specific manner to a complementary nucleic acid molecule (e.g., a nucleic acid molecule comprising a target sequence). The term “primer”, in particular, generally refers to an oligonucleotide that acts as a point of initiation of a template-directed synthesis using methods such as PCR (polymerase chain reaction) or LCR (ligase chain reaction) under appropriate conditions (e.g., in the presence of four different nucleotide triphosphates and a polymerization agent, such as DNA polymerase, RNA polymerase or reverse-transcriptase, DNA ligase, etc, in an appropriate buffer solution containing any necessary co-factors and at a suitable temperature). Such a template directed synthesis is also called “primer extension”. For example, a primer pair may be designed to amplify a region of DNA using PCR. Such a pair will include a “forward primer” and a “reverse primer” that hybridize to complementary strands of a DNA molecule and that delimit a region to be synthesized/amplified.

Typically, an oligonucleotide probe or primer will comprise a region of nucleotide sequence that hybridizes to at least about 8, more preferably at least about 10 to about 15, typically about 20 to about 40 consecutive nucleotides of a target nucleic acid (i.e., will hybridize to a contiguous sequence of the target nucleic acid). Oligonucleotides that exhibit differential or selected binding to a polymorphic site may readily be designed by one of ordinary skill in the art. For example, an oligonucleotide that is perfectly complementary to a sequence that encompasses a polymorphic site will hybridize to a nucleic acid comprising that sequence as opposed to a nucleic acid comprising an alternate polymorphic variant.

The terms “forward primer” and “forward amplification primer” are used herein interchangeably, and refer to a primer that hybridizes (or anneals) to the target (template strand). The terms “reverse primer” and “reverse amplification primer” are used herein interchangeably, and refer to a primer that hybridizes (or anneals) to the complementary target strand. The forward primer hybridizes with the target sequence 5′ with respect to the reverse primer.

The terms “probe” and “detection probe” are used herein interchangeably and refer to an oligonucleotide capable of selectively hybridizing to at least a portion of a target sequence under appropriate conditions. In general, a probe sequence is identified as being either “complementary” (i.e., complementary to the coding or sense strand (+)), or “reverse complementary” (i.e., complementary to the anti-sense strand (−)). A detection probe may be labeled with a detectable moiety.

As used herein, the term “allele-specific primer” refers to a primer whose 3′-terminal base is complementary to the corresponding template base for a particular allele at the polymorphic site. An allele-specific primer may comprise a sequence that is perfectly complementary to a sequence of the template immediately upstream to the polymorphic site. The term “allele-specific primer extension or ASPE” refers to a process in which an oligonucleotide primer is annealed to a DNA template 3′ with respect to a nucleotide indicative of the presence or absence of a target allele, and then extended in the presence of dNTP (e.g., labeled dNTP).

The term “amplification conditions”, as used herein, refers to conditions that promote annealing and/or extension of primer sequences. Such conditions are well-known in the art and depend on the amplification method selected. Thus, for example, in a PCR reaction, amplification conditions generally comprise thermal cycling, i.e., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and temperature cycling, buffer, salt, ionic strength, and pH, and the like.

As used herein, the term “amplification reaction reagents”, refers to reagents used in nucleic acid amplification reactions and may include, but are not limited to, buffers, reagents, enzymes having reverse transcriptase and/or polymerase activity or exonuclease activity, enzyme cofactors such as magnesium or manganese, salts, nicotinamide adenine dinuclease (NAD) and deoxynucleoside triphosphates (dNTPs), such as deoxyadenosine triphosphate, deoxyguanosine triphosphate, deoxycytidine triphosphate and deoxythymidine triphosphate. Amplification reaction reagents may readily be selected by one skilled in the art depending on the amplification method used.

The term “multiplex PCR reaction” refers to a PCR reaction in which multiple PCR amplifications are performed simultaneously in a single vessel or container and in which a plurality of (i.e., at least 2) amplification products are generated using a plurality of primer pairs. A collection of primer pairs is suitable for use in a multiplex PCR reaction if each of the primer pairs generates a discrete amplification product under at least one set of PCR conditions, without significant interference and/or cross-reactivity by one or more members of the other primer pairs present in the multiplex PCR reaction. In certain embodiments, PCR conditions of a multiplex PCR reaction may be optimized to compensate for the particular polymerase used, particular nucleic acid sequences, polypeptides, small molecules, metabolites, inorganic ions and/or other factors present in the reaction mixture, the method or methods used to isolate the nucleic acid for amplification, and/or any of a variety of other conditions known to those of ordinary skill in the art that may affect the PCR including, but not limited to, the efficacy, fidelity, or speed of the polymerization reaction.

The term “active fragment”, as used herein in reference to an oligonucleotide (e.g., an oligonucleotide sequence provided herein), refers to any nucleic acid molecule comprising a nucleotide sequence sufficiently homologous to or derived from the nucleotide sequence of the oligonucleotide, which includes fewer nucleotides than the full length oligonucleotide, and retains at least one biological property of the entire sequence. Typically, active fragments comprise a sequence with at least one activity of the full length oligonucleotide. An active fragment or portion of an oligonucleotide sequence of the present invention can be a nucleic acid molecule which is, for example, 10, 15, 20, 25, 30 or more nucleotides in length and can be used as amplification primer and/or detection probe for the detection of at least one CYP2D6 polymorphism in a sample.

The term “sufficiently homologous”, when used herein in reference to an active fragment of an oligonucleotide, refers to a nucleic acid molecule that has a sequence homology of at least 35% compared to the oligonucleotide. In certain embodiments, the sequence homology is at least 40%, at least 50%, at least 60%, at least 70%, at least 80%, at least 90%, at least 95%, or more than 95%.

The terms “homology” and “identity” are used herein interchangeably, and refer to the sequence similarity between two nucleic acid molecules. Calculation of the percent homology or identity of two nucleic acid sequences can be performed by aligning the two sequences for optimal comparison purposes (e.g., gaps can be introduced in one or both of a first and a second nucleic acid sequence for optimal alignment and non-homologous sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or more than 95% (e.g., 99%, or 100%) of the length of the reference sequence. The nucleotides at corresponding nucleotide positions are then compared. When a position in the first sequence is occupied by the same nucleotide as the corresponding position in the second sequence, then the molecules are identical (or homologous) at that position. The percent identify between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0), using a PAM120 weight residue table, a gap length penalty of 12 and a gap penalty of 4. The percent identity between two nucleotide sequences can, alternatively, be determined using the GAP program in the GCG software package (available at http://www.gcg.com), using a NWSgapdna.CMP matrix.

The terms “labeled” and “labeled with a detectable agent (or moiety)” are used herein interchangeably to specify that an entity (e.g., a target sequence) can be visualized, for example following hybridization to another entity (e.g., a probe). Preferably, the detectable agent or moiety is selected such that it generates a signal which can be measured and whose intensity is related to (e.g., proportional to) the amount of hybrid. Methods for labeling nucleic acid molecules are well-known in the art. Labeled nucleic acids can be prepared by incorporation of, or conjugation to, a label that is directly or indirectly detectable by spectroscopic, photochemical, biochemical, immunochemical, electrical, optical, or chemical means. Suitable detectable agents, include, but are not limited to, radionuclides, fluorophores, chemiluminescent agents, microparticles, enzymes, colorimetric labels, magnetic labels, haptens, molecular beacons, and aptamer beacons.

The term “fluorophore”, “fluorescent moiety”, and “fluorescent dye” are used herein interchangeably. They refer to a molecule that absorbs a quantum of electromagnetic radiation at one wavelength, and emits one or more photons at a different, typically longer wavelength in response. Numerous fluorescent dyes of a wide variety of structures and characteristics are suitable for use in the practice of the present invention. Methods and materials are known for fluorescently labeling nucleic acid molecules (see, for example, R. P. Haugland, “Molecular Probes: Handbook of Fluorescent Probes and Research Chemicals 1992-1994”, 5^{th} Ed., 1994, Molecular Probes, Inc.). Rather than being directly detectable themselves, some fluorescent dyes transfer energy to another fluorescent dye in a process of non-radiative fluorescence resonance energy transfer (FRET), and the second dye produces the detected signal. Such FRET fluorescent dye pairs are also encompassed by the term “fluorescent moiety”. The use of physically linked fluorescent reporter/quencher molecule is also within the scope of the invention. In these embodiments, when the reporter and quencher moieties are held in close proximity, such as at the ends of a nucleic acid probe, the quencher moiety prevents detection of a fluorescent signal from the reporter moiety. When the two moieties are physically separated, for example in the absence of target, the fluorescence signal from the reporter moiety becomes detectable.

As used herein, the term “diagnostic information” refers to any information that is useful in determining whether a patient has or is susceptible to develop a disease or condition and/or in classifying the disease or condition into a phenotypic category or any category having significance with regards to the prognostic or severity of, or likely response to treatment (either treatment in general or any particular treatment) of the disease or condition. Diagnostic information can include, for example, an assessment of the likelihood that an individual will suffer an adverse drug reaction if treated with a typical dose of a particular drug. Diagnostic information includes any information useful in selecting an appropriate regimen, e.g., drug, drug dose, dosing interval, etc. In the context of the present invention, “diagnosis” refers to providing any type of diagnostic information, including, but not limited to, whether a subject has a particular CYP2D6 allele, whether a subject is an extensive, poor, intermediate, or ultra-rapid metabolizer of drugs that are, at least in part, metabolized by CYP2D6, whether a subject is at increased risk of suffering an adverse drug reaction relative to an individual having a “wild-type” CYP2D6 genotype, or whether a subject is at increased risk of developing a particular disease relative to an individual having a “wild-type” CYP2D6 genotype.

The term “microparticle” is used herein to refer to particles having a smallest cross-sectional dimension of 50 microns or less. For example, the smallest cross-sectional dimension may be approximately 10 microns or less, approximately 3 microns or less, approximately 1 micron or less, or approximately 0.5 microns or less, e.g., approximately 0.1, 0.2, 0.3 or 0.4 microns. Microparticles may be made of a variety of inorganic or organic materials including, but not limited to, glass (e.g., controlled pore glass), silica, zirconia, cross-linked polystyrene, polyacrylate, polymethylmethacrylate, titanium dioxide, latex, polystyrene, etc. See, for example, U.S. Pat. No. 6,406,848 for various suitable materials and other considerations. Magnetically responsive microparticles can be used. Luminex xMAP microspheres, from Luminex (Austin, Tex.), are an example of commercially available microparticles suitable for use in the present invention. In certain embodiments, one or more populations of fluorescent microparticles are employed. The populations may have different fluorescence characteristics so that they can be distinguished from one another, e.g., using flow cytometry. In some embodiments, the microparticles are modified, e.g., an oligonucleotide is attached to a microparticle to serve as a “zip code” that allows specific hybridization to a second oligonucleotide that comprises a portion that is complementary to the zip code as described in more detail elsewhere herein.

### DETAILED DESCRIPTION OF CERTAIN PREFERRED EMBODIMENTS

As mentioned above, the present invention relates to methods and reagents for selectively detecting the presence of allelic variants of cytochrome P450 2D6 (CYP2D6) gene and determining their identity. In certain embodiments, the inventive methods use CYP2D6-specific oligonucleotide sequences and sensitive nucleic acid amplification-based techniques that allow for the detection of clinically relevant CYP2D6 polymorphisms, in particular, CYP2D6 polymorphisms associated with response to drugs or other xenobiotic compounds.

### I—Oligonucleotide Sequences for Amplification Primers and Detection Probes

### Inventive Oligonucleotide Sequences

CYP2D6 has more than 100 allelic variants currently identified (M. K. Ma et al., Am. J. Health Syst. Pharm., 2002, 59: 2061-2069; http://www.imm.ki.se/cypalleles/). These variants result from point mutations, deletions or additions, gene rearrangements, and deletion or duplication of the entire gene, and result in increase, reduction or complete loss of activity (M. K. Ma et al., Am. J. Health Syst. Pharm., 2002, 59: 2061-2069; M. Ingelman-Sundberg et al., Pharmacogenetics, 2001, 11: 553-554; M. Kitada, Int. J. Clin. Pharmacol. Res., 2003, 23: 31-35; H.-G. Xie et al., Annu Rev. Pharmacol. Toxicol., 2001, 41: 815-850).

The present invention provides oligonucleotide sequences that can be used in nucleic acid amplification tests for the detection of CYP2D6 polymorphisms. These nucleotide sequences are specific for the CYP2D6 gene and do not significantly cross-react with the pseudogenes CYP2D6 and CYP2D8, which are 97% homologous to CYP2D6. More specifically, oligonucleotide sequences are provided herein that can be used as amplification primers and detection probes to detect and identify CYP2D6 polymorphisms, including single nucleotide polymorphisms (SNPs), CYP2D6 gene duplication and CYP2D6 gene deletion.

In particular, oligonucleotide sequences are provided that can be used to amplify portions of CYP2D6 that contain SNPs. More specifically, oligonucleotide sequences of the invention can be used to amplify a target sequence that encompasses nucleotides 5173 to 8953 of the CYP2D6 gene (Accession NG_{—}003180). Products of this amplification reaction are herein called Amplicon A. Other oligonucleotide sequences of the invention can be used to amplify a target sequence that encompasses nucleotides 2922 to 4730 of the CYP2D6 gene (Accession M_{—}33388). Products of this amplification reaction are herein called Amplicon B.

In addition, oligonucleotide sequences are provided that can amplify a target sequence that encompasses nucleotide sequences of the truncated gene CYP2D7-CYP2D6 intergenic region (Accession x90926) and CYP2D6 (nucleotides 9361-9432, Accession M_{—}33388); from nucleotide 138 of gene CYP2D7-CYP2D6 intergenic region (Accession x90926) to nucleotide 9379 of CYP2D6 (Accession M_{—}33388), or from nucleotide 423 of gene CYP2D7-CYP2D6 intergenic region (Accession x90926) to nucleotide 9379 of CYP2D6 (Accession M_{—}33388); or from nucleotide 423 of gene CYP2D7-CYP2D6 intergenic region (Accession x90926) to nucleotide 9386 of CYP2D6 (Accession M_{—}33388) only if the sample tested contains a CYP2D6 gene deletion. Products of this amplification reaction are herein called Amplicon C. The present invention also provides oligonucleotide sequences that can amplify a target sequence that encompasses nucleotide sequences of truncated gene of CYP2D6: from nucleotides 1-9250 (Accession M_{—}33388) to nucleotides 3461-4200 (Accession NG_{—}003180); from nucleotide 6308 of CYP2D6 (Accession M_{—}33388) to nucleotide 3748 of CYP2D6 (Accession NG_{—}003180) only if the sample tested contains a CYP2D6 duplication. Products of this amplification are herein called Amplicon D.

Exemplary CYP2D6-specific oligonucleotide sequences for amplification primers provided by the present invention are presented in Table 1. In particular, Amplicon A (as described above) can be produced by PCR using a forward primer comprising SEQ ID NO. 1 and a reverse primer comprising SEQ ID NO. 2. Amplicon B can be produced by PCR using a forward primer comprising SEQ ID NO. 3 and a reverse primer comprising SEQ ID No. 4. Amplicon C can be produced by PCR using a forward primer comprising SEQ ID NO. 5 and a reverse primer comprising SEQ ID NO. 7; or a forward comprising SEQ ID NO. 6 and a reverse primer comprising SEQ ID NO. 7; or a forward primer comprising SEQ ID NO. 6 and a reverse primer comprising SEQ ID NO. 8. Amplicon D can be produced by PCR using a forward primer comprising SEQ ID NO. 9 and a reverse primer comprising SEQ ID NO. 10.

The present invention further provides oligonucleotide sequences that can be used as detection probes to detect and identify different SNPs located within Amplicon A and Amplicon B, as well as oligonucleotide sequences that can be used as detection probes to detect CYP2D6 gene duplication and CYP2D6 gene deletion.

Exemplary oligonucleotide sequences of the present invention that can be used as detection probes are presented in Table 2. In particular, these oligonucleotide sequences can be used to detect CYP2D6 duplication, CYP2D6 deletion (*5) and the following twelve (12) SNPs of CYP2D6: −1584 C>G (*2A); 124 G>A (*12); 100 C>T (*4, *10); 883 G>C (*11); 1023 C>T (*17); 1707 T>del (*6); 1758 G>T (*8); 1846 G>A (*4); 2549 A>del (*3); 2613-1615 del AGA (*9); 2850 C>T (*2, *17); and 2935 A>C (*7). Alternatively or additionally, these nucleic acid sequences can be used to identify additional SNPs of CYP2D6 of clinical interest that reside within Amplicon A and Amplicon B. Examples of such additional SNPs include: 138 ins T (*15); 1716 G>A (*7); 1716 G>A (*45, *46); 2539-2542 del AACT (*19); and 2573 ins C (*5).

In particular, CYP2D6 deletion can be detected using probes comprising SEQ ID NO. 84 and SEQ ID NO. 85, which hybridize to Amplicon C. CYP2D6 duplication can be detected using probes comprising SEQ ID NO. 86, SEQ ID NO. 87 and SEQ ID NO 88, which hybridize to Amplicon D. Wild-type (W) and mutant (M) probes comprising SEQ ID NOs. 11 through 83 can be used in Allele Specific Primer Extension (ASPE) reactions to specifically detect SNPs within Amplicon A and Amplicon B. SNPs within Amplicon A that can be detected according to the present invention include the following 5 SNPs: *2A, *12, *10, *11, and *17; SNPs within Amplicon B that can be detected according to the present invention include the following 7 SNPs: *6, *8, *4, *3, *9, *2, and *7.

More specifically, a wild-type probe comprising SEQ ID NO. 11 and a mutant probe comprising SEQ ID NO. 12 can be used to detect −1584 C>G. Wild-type and mutant probes comprising SEQ ID NOs. 13-24 can be used to detect 100 C>T. Wild-type and mutant probes comprising SEQ ID NOs. 25-32 can be used to detect 124 G>A. Wild-type and mutant probes comprising SEQ ID NOs. 33-36 can be used to detect 883 G>C. Wild-type and mutant probes comprising SEQ ID Nos. 37-44 can be used to detect 1023 C>T. Wild-type and mutant probes comprising SEQ ID NOs. 45-52 can be used to detect 1707 T>del. Wild-type and mutant probes comprising SEQ ID NOs. 53-56 can be used to detect 1758 G>T. Wild-type and mutant probes comprising SEQ ID NOs. 57-65 can be used to detect 1846 G>A. Wild-type and mutant probes comprising SEQ ID NOs. 66 and 67 can be used to detect 2549 A>del. Wild-type and mutant probes comprising SEQ ID NOs. 68-75 can be used to detect 2613-1615 del AGA. Wild-type and mutant probes comprising SEQ ID NOs. 76-81 can be used to detect 2850 C>T. Wild-type and mutant probes comprising SEQ ID NOs. 82 and 83 can be used to detect 2935 A>C. It is within the expertise of one skilled in the art to select suitable wild-type and mutant probes provided herein for the detection of a particular CYP2D6 SNP.

As will be appreciated by one skilled in the art, some of the oligonucleotide sequences of the present invention may be employed either as amplification primers or detection probes depending on the intended use or assay format. For example, an inventive oligonucleotide sequence used as an amplification primer in one assay can be used as a detection probe in a different assay. A given sequence may be modified, for example, by attaching to the inventive oligonucleotide sequence, a specialized sequence (e.g., a promoter sequence) required by the selected amplification method, or by attaching a fluorescent dye to facilitate detection. It is also understood that an oligonucleotide according to the present invention may include one or more sequences which serve as spacers, linkers, sequences of labeling or binding to an enzyme, which may impart added stability or susceptibility to degradation process or other desirable property to the oligonucleotide.

Based on the oligonucleotide sequences provided herein, one or more oligonucleotide analogues can be prepared (see below). Such analogues may contain alternative structures such as peptide nucleic acids or “PNAs” (i.e., molecules with a peptide-like backbone instead of the phosphate sugar backbone of naturally-occurring nucleic acids) and the like. These alternative structures, representing the sequences of the present invention, are likewise part of the present invention. Similarly, it is understood that oligonucleotide consisting of the sequences of the present invention may contain deletions, additions, and/or substitutions of nucleic acid bases, to the extent that such alterations do not negatively affect the properties of the nucleic acid molecules. In particular, the alterations should not result in significant lowering of the hybridizing properties of the oligonucleotides.

### Primer Sets and Primer/Probe Sets

Primers and/or probes of the present invention may be conveniently provided in sets, e.g., sets capable of determining which polymorphic variant(s) is/are present among some or all of the possible polymorphic variants that may exist at a particular polymorphic site. Multiple sets of primers and/or probes, capable of detecting polymorphic variants at a plurality of polymorphic sites may be provided.

As used herein, the term “primer set” refers to two or more primers which together can be used to prime the amplification of a nucleotide sequence of interest (e.g., to generate Amplicon A, Amplicon B, Amplicon C or Amplicon D). In certain embodiments, the term “primer set” refers to a pair of primers including a 5′ (upstream) primer (or forward primer) that hybridizes with the 5′-end of the nucleic acid sequence to be amplified and a 3′ (downstream) primer (or reverse primer) that hybridizes with the complement of the sequence to be amplified. Such primer sets or primer pairs are particularly useful in PCR amplification reactions.

Examples of primer sets/pairs comprising a forward amplification primer and a reverse amplification primer include: Primer Set 1, which comprises a forward primer comprising SEQ ID NO. 1, or any active fragment thereof, and a reverse primer comprising SEQ ID NO. 2, or any active fragment thereof; Primer Set 2, which comprises a forward primer comprising SEQ ID NO. 3, or any active fragment thereof, and a reverse primer comprising SEQ ID NO. 4, or any active fragment thereof; Primer Set 3, which comprises a forward primer comprising SEQ ID NO. 5, or any active fragment thereof, and a reverse primer comprising SEQ ID NO. 7, or any active fragment thereof; Primer Set 4, which comprises a forward primer comprising SEQ ID NO. 6, or any active fragment thereof, and a reverse primer comprising SEQ ID NO. 7, or any active fragment thereof; Primer Set 5, which comprises a forward primer comprising SEQ ID NO. 6, or any active fragment thereof, and a reverse primer comprising SEQ ID NO. 8, or any active fragment thereof and Primer Set 6, which comprises a forward primer comprising SEQ ID NO. 9, or any active fragment thereof, and a reverse primer comprising SEQ ID NO. 10, or any active fragment thereof.

In addition to primer sets, the present invention provides probe sets. As used herein, the term “probe set” refers to two or more probes which together allow detection of at least one CYP2D6 polymorphisms of interest (e.g., a SNP located within Amplicon A or Amplicon B). In certain embodiments, the term “primer set” refers to a pair of allele-specific oligonucleotides (one wild type (W) and one mutant (M) probes) that can be used in an ASPE reaction to detect a SNP of interest. It is within the expertise of one skilled in the art to select suitable wild-type and mutant probes provided herein to form a probe set for the detection of a particular SNP.

The present invention further provides primer/probe sets. As used herein, the term “primer/probe set” refers to a combination comprising two or more primers which together are capable of priming the amplification of a CYP2D6 nucleotide sequence of interest to generate an amplification product (e.g., Amplicon A, Amplicon B, Amplicon C, or Amplicon D), and two or more probes which together allow detection of at least one CYP2D6 polymorphism associated with the amplification product (e.g., a SNP within Amplicon A). In certain embodiments, the term “primer/probe set” refers to a pair of forward primer and reverse primer that generate an amplification product of interest by PCR and at least one pair of allele-specific oligonucleotides (one wild-type probe and one mutant probe) that can be used in an ASPE reaction to detect a SNP that resides within the amplification product obtained by PCR. Several primer/probe sets may be used (for example assembled in a kit) for multiplex detection of CYP2D6 polymorphisms.

### Oligonucleotide Preparation

Oligonucleotides of the invention may be prepared by any of a variety of methods (see, for example, J. Sambrook et al., “Molecular Cloning: A Laboratory Manual”, 1989, 2^{nd} Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5^{th} Ed., John Wiley & Sons: Secaucus, N.J.). For example, oligonucleotides may be prepared using any of a variety of chemical techniques well-known in the art, including, for example, chemical synthesis and polymerization based on a template as described, for example, in S. A. Narang et al., Meth. Enzymol. 1979, 68: 90-98; E. L. Brown et al., Meth. Enzymol. 1979, 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; and U.S. Pat. No. 4,458,066.

Oligonucleotides may be prepared using an automated, solid-phase procedure based on the phosphoramidite approach. In such a method, each nucleotide is individually added to the 5′-end of the growing oligonucleotide chain, which is attached at the 3′-end to a solid support. The added nucleotides are in the form of trivalent 3′-phosphoramidites that are protected from polymerization by a dimethoxytriyl (or DMT) group at the 5′-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on oligo synthesizers such as those commercially available from Perkin Elmer/Applied Biosystems, Inc. (Foster City, Calif.), DuPont (Wilmington, Del.) or Milligen (Bedford, Mass.). Alternatively, oligonucleotides can be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), and many others.

Purification of the oligonucleotides of the invention, where necessary, may be carried out by any of a variety of methods well-known in the art. Purification of oligonucleotides is typically performed either by native acrylamide gel electrophoresis, by anion-exchange HPLC as described, for example, by J. D. Pearson and F. E. Regnier (J. Chrom., 1983, 255: 137-149) or by reverse phase HPLC (G. D. McFarland and P. N. Borer, Nucleic Acids Res., 1979, 7: 1067-1080).

The sequence of oligonucleotides can be verified using any suitable sequencing method including, but not limited to, chemical degradation (A. M. Maxam and W. Gilbert, Methods of Enzymology, 1980, 65: 499-560), matrix-assisted laser desorption ionization time-of-flight (MALDI-TOF) mass spectrometry (U. Pieles et al., Nucleic Acids Res., 1993, 21: 3191-3196), mass spectrometry following a combination of alkaline phosphatase and exonuclease digestions (H. Wu and H. Aboleneen, Anal. Biochem., 2001, 290: 347-352), and the like.

As already mentioned above, modified oligonucleotides may be prepared using any of several means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc), or charged linkages (e.g., phosphorothioates, phosphorodithioates, etc). Oligonucleotides may contain one or more additional covalently linked moieties, such as, for example, proteins (e.g., nucleases, toxins, antibodies, signal peptides, poly-L-lysine, etc), intercalators (e.g., acridine, psoralen, etc), chelators (e.g., metals, radioactive metals, iron, oxidative metals, etc), and alkylators. The oligonucleotide may also be derivatized by formation of a methyl or ethyl phosphotriester or an alkyl phosphoramidate linkage. Furthermore, the oligonucleotide sequences of the present invention may also be modified with a label.

### Labeling of Oligonucleotide Sequences

In certain embodiments, the detection probes or amplification primers or both probes and primers are labeled with a detectable agent or moiety before being used in amplification/detection assays. In certain embodiments, the detection probes are labeled with a detectable agent. For example, a wild-type probe and mutant probe to be used for the ASPE-based detection of a SNP of interest may be labeled with two different detectable agents to allow for identification of the SNP. Preferably, a detectable agent is selected such that it generates a signal which can be measured and whose intensity is related (e.g., proportional) to the amount of amplification products in the sample being analyzed.

The association between the oligonucleotide and detectable agent can be covalent or non-covalent. Labeled detection probes can be prepared by incorporation of or conjugation to a detectable moiety. Labels can be attached directly to the nucleic acid sequence or indirectly (e.g., through a linker). Linkers or spacer arms of various lengths are known in the art and are commercially available, and can be selected to reduce steric hindrance, or to confer other useful or desired properties to the resulting labeled molecules (see, for example, E. S. Mansfield et al., Mol. Cell. Probes, 1995, 9: 145-156).

Methods for labeling nucleic acid molecules are well-known in the art. For a review of labeling protocols, label detection techniques, and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachments of fluorescent dyes (L. M. Smith et al., Nucl. Acids Res., 1985, 13: 2399-2412) or of enzymes (B. A. Connoly and O. Rider, Nucl. Acids. Res., 1985, 13: 4485-4502); chemical modifications of nucleic acid molecules making them detectable immunochemically or by other affinity reactions (T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl. Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232). More recently developed nucleic acid labeling systems include, but are not limited to: ULS (Universal Linkage System), which is based on the reaction of mono-reactive cisplatin derivatives with the N7 position of guanine moieties in DNA (R. J. Heetebrij et al., Cytogenet. Cell. Genet. 1999, 87: 47-52), psoralen-biotin, which intercalates into nucleic acids and upon UV irradiation becomes covalently bonded to the nucleotide bases (C. Levenson et al., Methods Enzymol. 1990, 184: 577-583; and C. Pfannschmidt et al., Nucleic Acids Res. 1996, 24: 1702-1709), photoreactive azido derivatives (C. Neves et al., Bioconjugate Chem. 2000, 11: 51-55), and DNA alkylating agents (M. G. Sebestyen et al., Nat. Biotechnol. 1998, 16: 568-576).

Any of a wide variety of detectable agents can be used in the practice of the present invention. Suitable detectable agents include, but are not limited to, various ligands, radionuclides (such as for example, ³²P, ³⁵S, ³H, ¹⁴C, ¹²⁵I, ¹³¹I, and the like); fluorescent dyes (for specific exemplary fluorescent dyes, see below); chemiluminescent agents (such as, for example, acridinium esters, stabilized dioxetanes, and the like); spectrally resolvable inorganic fluorescent semiconductor nanocrystals (i.e., quantum dots), metal nanoparticles (e.g., gold, silver, copper and platinum) or nanoclusters; enzymes (such as, for example, those used in an ELISA, i.e., horseradish peroxidase, beta-galactosidase, luciferase, alkaline phosphatase); colorimetric labels (such as, for example, dyes, colloidal gold, and the like); magnetic labels (such as, for example, Dynabeads™); and biotin, dioxigenin or other haptens and proteins for which antisera or monoclonal antibodies are available.

In certain embodiments, the inventive detection probes are fluorescently labeled. Numerous known fluorescent labeling moieties of a wide variety of chemical structures and physical characteristics are suitable for use in the practice of this invention. Suitable fluorescent dyes include, but are not limited to, fluorescein and fluorescein dyes (e.g., fluorescein isothiocyanine or FITC, naphthofluorescein, 4′,5′-dichloro-2′,7′-dimethoxy-fluorescein, 6-carboxyfluorescein or FAM), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™ cyanine dyes (e.g., Cy-3™, Cy-S™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. For more examples of suitable fluorescent dyes and methods for linking or incorporating fluorescent dyes to nucleic acid molecules see, for example, “The Handbook of Fluorescent Probes and Research Products”, 9^{th} Ed., Molecular Probes, Inc., Eugene, Oreg. Fluorescent dyes as well as labeling kits are commercially available from, for example, Amersham Biosciences, Inc. (Piscataway, N.J.), Molecular Probes Inc. (Eugene, Oreg.), and New England Biolabs Inc. (Berverly, Mass.).

Rather than being directly detectable themselves, some fluorescent groups (donors) transfer energy to another fluorescent group (acceptor) in a process of fluorescent resonance energy transfer (FRET), and the second group produces the detected fluorescent signal. In these embodiments, the oligonucleotide detection probe may, for example, become detectable when hybridized to an amplified target sequence. Examples of FRET acceptor/donor pairs suitable for use in the present invention include, but are not limited to fluorescein/tetramethylrhodamine, IAEDANS/FITC, IAEDANS/5-(iodoacetomido)fluorescein, EDANS/Dabcyl, and B-phyco-erythrin/Cy-5.

The use of physically linked fluorescent reporter/quencher molecule pairs is also encompassed within the scope of the invention. The use of such systems in TaqMan™ assays (as described, for example, in U.S. Pat. Nos. 5,210,015; 5,804,375; 5487,792 and 6,214,979) or as Molecular Beacons (as described, for example in, S. Tyagi and F. R. Kramer, Nature Biotechnol. 1996, 14: 303-308; S. Tyagi et al., Nature Biotechnol. 1998, 16: 49-53; L. G. Kostrikis et al., Science, 1998, 279: 1228-1229; D. L. Sokol et al., Proc. Natl. Acad. Sci. USA, 1998, 95: 11538-11543; S. A. Marras et al., Genet. Anal. 1999, 14: 151-156; and U.S. Pat. Nos. 5,846,726, 5,925,517, 6,277,581 and 6,235,504) is well-known in the art.

A “tail” of normal or modified nucleotides can also be added to oligonucleotide probes for detectability purposes. A second hybridization with nucleic acid complementary to the tail and containing one or more detectable labels (such as, for example, fluorophores, enzymes or bases that have been radioactivity labeled or microparticles) allows visualization of the amplicon/probe hybrids (see, for example, the system commercially available from Enzo Biochem. Inc., New York: NY). Another example of an assay with which the inventive oligonucleotides are useful is a signal amplification method such as that described in U.S. Pat. No. 5,124,246 (which is incorporated herein by reference in its entirety). In that method, the signal is amplified through the use of amplification multimers, polynucleotides which are constructed so as to contain a first segment that hybridizes specifically to the “tail” added to the oligonucleotide probes, and a multiplicity of identical second segments that hybridize specifically to a labeled probe. The degree of amplification is theoretically proportional to the number of iterations of the second segment. The multimers may be either linear or branched. Branched multimers may be in the shape of a fork or a comb.

The selection of a particular nucleic acid labeling technique will depend on the situation and will be governed by several factors, such as the ease and cost of the labeling method, the quality of sample labeling desired, the effects of the detectable moiety on the hybridization reaction (e.g., on the rate and/or efficiency of the hybridization process), the nature the of amplification method used, the nature of the detection system, the nature and intensity of the signal generated by the detectable label, and the like.

### II—Detection of CYP2D6 Polymorphisms

As already mentioned above, the oligonucleotide sequences of the present invention can be used in nucleic acid amplification methods for detecting the presence and identifying CYP2D6 polymorphisms in a test sample obtained from an individual.

Detection methods of the present invention will generally include: preparation of a test sample comprising the CYP2D6 gene or genetic material comprising the CYP2D6 gene; amplification of at least one CYP2D6 target sequence using amplification primers provided herein (e.g., by PCR using a forward primer and a reverse primer) to produce an amplification products (e.g., Amplicon A, Amplicon B, Amplicon C and/or Amplicon D); and detection of at least one CYP2D6 polymorphism associated with amplification products using detection probes provided herein (e.g., by ASPE using wild-type and mutant probes).

### Sample Preparation

Test samples suitable for use in detection methods of the present invention contain genetic material, i.e., DNA. Such DNA may be obtained from any cell source. Non-limiting examples of cell sources in clinical practice include, blood cells, buccal cells, cervico-vaginal cells, epithelial cells from urine, fetal cells, or any cells present in tissue obtained by biopsy. Cells may be obtained from body fluids (e.g., blood, serum, urine, sputum, saliva, cerebrospinal fluid, seminal fluid, lymph fluid, and the like), or tissues (e.g., skin, hair, buccal or conjunctival mucosa, muscles, bone marrow, lymph nodes, and the like). DNA from fetal or embryonic cells or tissues can be obtained by appropriate methods, such as amniocentesis or chorionic villus sampling.

Isolation, extraction or derivation of DNA may be carried out by any suitable method. Isolating DNA from a biological sample generally includes treating a biological sample in such a manner that genomic DNA present in the sample is extracted and made available for analysis. Any isolation method that results in extracted genomic DNA may be used in the practice of the present invention. It will be understood that the particular method used to extract DNA will depend on the nature of the source.

Methods of DNA extraction are well-known in the art. A classical DNA isolation protocol is based on extraction using organic solvents such as a mixture of phenol and chloroform, followed by precipitation with ethanol (J. Sambrook et al., “Molecular Cloning: A Laboratory Manual”, 1989, 2^{nd} Ed., Cold Spring Harbour Laboratory Press: New York, N.Y.). Other methods include: salting out DNA extraction (P. Sunnucks et al., Genetics, 1996, 144: 747-756; S. M. Aljanabi and I. Martinez, Nucl. Acids Res. 1997, 25: 4692-4693), trimethylammonium bromide salts DNA extraction (S. Gustincich et al., BioTechniques, 1991, 11: 298-302) and guanidinium thiocyanate DNA extraction (J. B. W. Hammond et al., Biochemistry, 1996, 240: 298-300).

There are also numerous versatile kits that can be used to extract DNA from tissues and bodily fluids and that are commercially available from, for example, BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

In certain embodiments, methods of the present invention are practiced on cellular material other than DNA. For example, polymorphisms that lie in the CYP2D6 gene may be detected in RNA.

Methods of RNA extraction are well known in the art (see, for example, J. Sambrook et al., “Molecular Cloning: A Laboratory Manual”, 1989, 2^{nd} Ed., Cold Spring Harbour Laboratory Press: New York) and several kits for RNA extraction from bodily fluids are commercially available, for example, from Ambion, Inc. (Austin, Tex.), Amersham Biosciences (Piscataway, N.J.), BD Biosciences Clontech (Palo Alto, Calif.), BioRad Laboratories (Hercules, Calif.), Dynal Biotech Inc. (Lake Success, N.Y.), Epicentre Technologies (Madison, Wis.), Gentra Systems, Inc. (Minneapolis, Minn.), GIBCO BRL (Gaithersburg, Md.), Invitrogen Life Technologies (Carlsbad, Calif.), MicroProbe Corp. (Bothell, Wash.), Organon Teknika (Durham, N.C.), Promega, Inc. (Madison, Wis.) and Qiagen Inc. (Valencia, Calif.).

Instead of being performed on extracted genetic material, detection methods of the present invention may be performed in situ directly upon tissue sections (fixed and/or frozen) of patient tissue obtained from biopsies or resection, such that no nucleic acid extraction/purification is necessary. Nucleic acid reagents may be used as probes and/or primers for such in situ procedures (see, for example, G. J. Nuova, “PCR in situ Hybridization: Protocols and Application”, 1992, Raven Press: NY).

### Amplification of CYP2D6 Target Sequences Using Inventive Primers

The use of oligonucleotide sequences of the present invention to amplify CYP2D6 target sequences in test samples is not limited to any particular nucleic acid amplification technique or any particular modification thereof. In fact, the inventive oligonucleotide sequences can be employed in any of a variety of nucleic acid amplification methods well-known in the art (see, for example, A. R. Kimmel and S. L. Berger, Methods Enzymol. 1987, 152: 307-316; J. Sambrook et al., “Molecular Cloning: A Laboratory Manual”, 1989, 2^{nd} Ed., Cold Spring Harbour Laboratory Press: New York, N.Y.; “Short Protocols in Molecular Biology”, F. M. Ausubel (Ed.), 2002, 5^{th} Ed., John Wiley & Sons: Secaucus, N.J.).

Such well-known nucleic acid amplification methods include, but are not limited to, the Polymerase Chain Reaction (or PCR, described, for example, in “PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; Saiki et al., Nature, 1986, 324: 163; and U.S. Pat. Nos. 4,683,195, 4,683,202 and 4,889,818, each of which is incorporated herein by reference in its entirety); and variations thereof including TaqMan™-based assays (Holland et al., Proc. Natl. Acad. Sci., 1991, 88: 7276-7280), and reverse transcriptase polymerase chain reaction (or RT-PCR, described in, for example, U.S. Pat. Nos. 5,322,770 and 5,310,652).

The PCR (or polymerase chain reaction) technique is well-known in the art and has been disclosed in K. B. Mullis and F. A. Faloona, Methods Enzymol., 1987, 155: 355-350 and U.S. Pat. Nos. 4,683,202; 4,683,195; and 4,800,159 (each of which is incorporated herein by reference in its entirety). In its simplest form, PCR is an in vitro method for the enzymatic synthesis of specific DNA sequences, using two oligonucleotide primers that hybridize to opposite strands and flank the region of interest in the target DNA. A plurality of reaction cycles, each cycle comprising: a denaturation step, an annealing step, and a polymerization step, results in the exponential accumulation of a specific DNA fragment (“PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; R. K. Saiki et al., Nature, 1986, 324: 163-166). The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). RNA target sequences may be amplified by reverse transcribing the mRNA into cDNA, and then performing PCR(RT-PCR), as described above. Alternatively, a single enzyme may be used for both steps as described in U.S. Pat. No. 5,322,770.

The duration and temperature of each step of a PCR cycle, as well as the number of cycles, are generally adjusted according to the stringency requirements in effect. Annealing temperature and timing are determined both by the efficiency with which a primer is expected to anneal to a template and the degree of mismatch that is to be tolerated. The ability to optimize the reaction cycle conditions is well within the knowledge of one of ordinary skill in the art. Although the number of reaction cycles may vary depending on the detection analysis being performed, it usually is at least 15, more usually at least 20, and may be as high as 60 or higher. However, in many situations, the number of reaction cycles typically ranges from about 20 to about 40.

The denaturation step of a PCR cycle generally comprises heating the reaction mixture to an elevated temperature and maintaining the mixture at the elevated temperature for a period of time sufficient for any double-stranded or hybridized nucleic acid present in the reaction mixture to dissociate. For denaturation, the temperature of the reaction mixture is usually raised to, and maintained at, a temperature ranging from about 85° C. to about 100° C., usually from about 90° C. to about 98° C., and more usually from about 93° C. to about 96° C. for a period of time ranging from about 3 to about 120 seconds, usually from about 5 to about 30 seconds.

Following denaturation, the reaction mixture is subjected to conditions sufficient for primer annealing to template DNA present in the mixture. The temperature to which the reaction mixture is lowered to achieve these conditions is usually chosen to provide optimal efficiency and specificity, and generally ranges from about 50° C. to about 75° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

Following annealing of primer to template DNA or during annealing of primer to template DNA, the reaction mixture is subjected to conditions sufficient to provide for polymerization of nucleotides to the primer's end in a manner such that the primer is extended in a 5′ to 3′ direction using the DNA to which it is hybridized as a template, (i.e., conditions sufficient for enzymatic production of primer extension product). To achieve primer extension conditions, the temperature of the reaction mixture is typically raised to a temperature ranging from about 65° C. to about 75° C., usually from about 67° C. to about 73° C., and maintained at that temperature for a period of time ranging from about 15 seconds to about 20 minutes, usually from about 30 seconds to about 5 minutes.

The above cycles of denaturation, annealing, and polymerization may be performed using an automated device typically known as a thermal cycler or thermocycler. Thermal cyclers that may be employed are described in U.S. Pat. Nos. 5,612,473; 5,602,756; 5,538,871; and 5,475,610 (each of which is incorporated herein by reference in its entirety). Thermal cyclers are commercially available, for example, from Perkin Elmer-Applied Biosystems (Norwalk, Conn.), BioRad (Hercules, Calif.), Roche Applied Science (Indianapolis, Ind.), and Stratagene (La Jolla, Calif.).

In addition to the enzymatic thermal amplification technique described above, well-known isothermal enzymatic amplification reactions can be employed to amplify CYP2D6 target sequences using oligonucleotide primers of the present invention (S. C. Andras et al., Mol. Biotechnol., 2001, 19: 29-44). These methods include, but are not limited to, Transcription-Mediated Amplification (or TMA, described in, for example, D. Y. Kwoh et al., Proc. Natl. Acad. Sci. USA, 1989, 86: 1173-1177; C. Giachetti et al., J. Clin. Microbiol., 2002, 40: 2408-2419; and U.S. Pat. No. 5,399,491); Self-Sustained Sequence Replication (or 3SR, described in, for example, J. C. Guatelli et al., Proc. Natl. Acad. Sci. USA, 1990, 87: 1874-1848; and E. Fahy et al., PCR Methods and Applications, 1991, 1: 25-33); Nucleic Acid Sequence Based Amplification (or NASBA, described in, for example, T. Kievits et al., J. Virol., Methods, 1991, 35: 273-286; and U.S. Pat. No. 5,130,238) and Strand Displacement Amplification (or SDA, described in, for example, G. T. Walker et al., PNAS, 1992, 89: 392-396; EP 0 500 224 A2). Each of the references cited in this paragraph is incorporated herein by reference in its entirety.

Amplification products obtained using primers of the present invention may be detected using agarose gel electrophoresis and visualization by ethidium bromide staining and exposure to ultraviolet (UV) light or by sequence analysis of the amplification product.

### Allele Specific Primer Extension Reaction

SNPs located within Amplicon A and Amplicon B can be specifically detected by allele-specific primer extension (ASPE) using detection probes comprising SEQ ID NOs. 11 through 83.

In ASPE, the presence or absence of a particular SNP is detected by selective amplification, wherein one of the alleles is amplified without amplification of the other allele(s). In these methods, allele-specific primers are used that anneal to the target and whose 3′-terminal base is complementary to the corresponding template base of one allele but is a mismatch for the alternative allele(s). Since the extension starts at the 3′-end of the primer, a mismatch at or near this position has an inhibitory effect on extension; and DNA polymerases extend primers with a mismatched 3′ nucleotide with a much lower efficiency that perfect matches. Therefore, under appropriate amplification conditions, only that allele which is complementary to the matched primer is amplified.

Methods of using allele-specific oligonucleotides, such as those described herein, have been extensively described (see, for example, C. R. Newton et al., Nucl. Acids Res., 1989, 17: 2503-2516; W. C. Nichols et al., Genomics, 1989, 5: 535-540; D. Y. Wu, Proc. Natl. Acad. Sci. USA, 1989, 86: 2757-2760; C Dutton and S. S. Sommer, Biotechniques, 1991, 11: 700-702; R. S. Cha et al., PCR Methods Appl., 1992, 2: 14-20; L. Ugozzoli and R. B. Wallace, Methods Enzymol., 1991, 2: 42-48).

As will be recognized by one skilled in the art, in the methods of the present invention, a single primer or a set of primers (e.g., forward and reverse primers) can be used depending on whether primer extension, linear or exponential amplification of the template is desired. When a single primer is used, the primer is typically an allele-specific primer, as described herein. When two primers are used, one is an allele-specific primer and the other is a complementary strand primer which anneals to the other DNA strand distant from the allele-specific primer. A set of primer pairs, wherein each pair comprises an allele-specific primer and a complementary strand primer, can also be used to distinguish alleles of a particular SNP. For example, the allele-specific primers of a set can be unique with respect to each other: one of the allele-specific primers may be complementary to the wild-type allele (i.e., allele-specific to the normal allele), and the others may be complementary to the alternative alleles. Each of the allele-specific primers in such a set may be paired with a common complementary strand primer. Multiple sets of pairs of primers can be used for the multiplex detection of SNPs.

In an ASPE reaction, amplification products (e.g., Amplicon A and/or Amplicon B obtained as described above) are generally combined with allele-specific primers (e.g., one wild-type oligonucleotide sequence and one mutant oligonucleotide sequence provided herein), deoxyribonucleoside triphosphates (dNTPs), a thermostable nucleic acid polymerase, and an aqueous buffer medium to form a primer extension reaction mixture.

The term “thermostable”, when used herein in reference to a nucleic acid polymerase, refers to an enzyme which is stable and active at a temperature as great as between about 90° C. and about 100° C., or between about 70° C. and about 98° C. A representative thermostable nucleic acid polymerase isolated from Thermus aquaticus (Taq) is described in U.S. Pat. No. 4,889,818 and a method for using it in conventional PCR is described in R. K. Saiki et al., Science, 1988, 239: 487-491. Another representative thermostable nucleic acid polymerase, isolated from P. furiosus (Pfu), is described in K. S. Lundberg et al., Gene, 1991, 108: 1-6. Additional examples of thermostable polymerases include polymerases extracted from the thermophilic bacteria Thermus flavus, Thermus ruber, Thermus thermophilus, Bacillus stearothermophilus, Thermus lacteus, Thermus rubens, Thermotoga maritima, or from thermophilic archaea Thermococcus litoralis and Methanothermus fervidus. Thermostable DNA polymerases suitable for use in the practice of the present invention include, but are not limited to, E. coli DNA polymerase I, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase.

In certain embodiments, the primer extension reaction mixture comprises a thermostable nucleic acid polymerase lacking 5′→3′ exonuclease activity or lacking both 5′→3′ and 3′→5′ exonuclease activity. With such nucleic acid polymerases, the target DNA is used as a template for extending the allele-specific oligonucleotide and no extension occurs if there is a mismatch at the terminal 3′ end of the allele-specific oligonucleotide.

Examples of nucleic acid polymerases substantially lacking 5′→3′ exonuclease activity include, but are not limited to, Klenow and Klenow exo-, and T7 DNA polymerase (Sequenase). Examples of thermostable nucleic acid polymerases substantially lacking 5′→3′ exonuclease activity include, but are not limited to, Pfu, the Stoffel fragment of Taq, N-truncated Bst, N-truncated Bca, Genta, JdF3 exo, Vent, Deep Vent, U1Tma and ThermoSequenase. Examples of thermostable nucleic acid polymerases substantially lacking both 5′→3′ and 3′→5′ exonuclease activity include, but are not limited to, exo-Pfu (a mutant form of Pfu), Vent exo (a mutant form of Vent), Deep Vent exo- (a mutant form of Deep Vent), and Platinum® GenoTYPE Tsp DNA polymerase. Thermostable nucleic acid polymerases are commercially available for example from Stratagene (La Jolla, Calif.), New England BioLabs (Ipswich, Mass.), BioRad (Hercules, Calif.), Perkin-Elmer (Wellesley, Mass.), Hoffman-LaRoche (Basel, Switzerland), and Invitrogen (Carlsbad, Calif.).

A primer extension reaction mixture generally comprises enough thermostable polymerase so that conditions suitable for enzymatic primer extension are maintained during all the reaction cycles. Alternatively, polymerase may be added to the primer extension reaction mixture after a certain number of reaction cycles have been performed.

The aqueous buffer medium generally acts as a source of monovalent ions, divalent cations, and buffer agent. Any convenient source of monovalent ions, such as potassium chloride, potassium acetate, potassium glutamate, ammonium acetate, ammonium chloride, ammonium sulfate, and the like may be employed. The divalent cation may be magnesium, manganese, zinc and the like. Magnesium (Mg²⁺) is often used. Any source of magnesium cations may be employed, including magnesium chloride, magnesium acetate, and the like. The amount of Mg²⁺ present in the buffer may range from about 0.5 to about 10 mM. Representative buffering agents, or salts that may be present in the buffer include Tris, Tricine, HEPES, MOPS, and the like. The amount of buffering agent generally ranges from about 5 to about 150 mM. In certain embodiments, the buffer agent is present in an amount sufficient to provide a pH ranging from about 6.0 to 9.5, most preferably pH 7.3. Other agents which may be present in the buffer medium include chelating agents, such as EDTA, EGTA and the like.

Generally, the primer extension reaction mixture will comprise four different types of dNTPs corresponding to the four naturally occurring bases, i.e., dATP, dTTP, dCTP, and dGTP. In certain embodiments, the primer extension mixture additionally contains biotinylated dNTPs, for example biotinylated dCTP, for incorporation of biotin in the primer extension product(s). The resulting biotinylated primer extension products may subsequently be exposed to a streptavidin-dye complex for detection purposes, as is well-known in the art. Examples of streptavidin-dye complexes suitable for use in the practice of the methods of the present invention include, but are not limited to, streptavidin-fluorescein (SA-FITC), streptavidin-phycoerythrin (SA-PE), streptavidin-rhodamine B (SA-R), streptavidin-Texas Red (SA-TR), streptavidin-phycocyanin (SA-PC), and streptavidin-allophycocyanine (SA-APC).

In preparing a primer extension reaction mixture, the various constituent components may be combined in any convenient order.

Following addition of all the components, the reaction mixture is subjected to primer extension reaction conditions, i.e., to conditions that allow for polymerase-mediated primer extension by addition of nucleotides to the end of the annealed (i.e., hybridized) primer molecule using the target strand as a template. In many embodiments, the primer extension reaction conditions are PCR amplification conditions (see above).

In the methods of the present invention, ASPE reactions may be performed under homogeneous or heterogeneous conditions. In a homogeneous ASPE reaction, all the reagents are in solution. Alternatively, detection probes capable of hybridizing specifically to allelic variants may be attached to a solid support. In some embodiments, such a solid support may be in the form of a chip or array. The solid support may be contacted with the PCR reaction mixture (e.g., containing Amplicon A and/or Amplicon B), and amplification products in the PCR reaction mixture are allowed to hybridize to one or more probes attached to the solid support. Primer extension may be performed after hybridization, as described above, for example using one or more labeled nucleotides. In other embodiments, each detection probe is attached to a microbead. The bead-labeled detection probes may be added to the PCR reaction mixture, and amplification products in the PCR reaction mixture are allowed to hybridize to one or more probes. Primer extension may be performed after hybridization, as described above.

### Detection of SNPs in Primer Extension Products

Analysis of primer extension products can be accomplished using any of a wide variety of methods.

Following primer extension performed under homogeneous conditions, it may be desirable to separate the primer extension products from each other and from other components of the reaction mixture (e.g., unamplified DNA, excess primers/probes, etc) for purpose of analysis. In certain embodiments, separation of primer extension products is accomplished by employing capture reagents. Capture reagents typically consist of a solid support material coated with one or more binding members specific for the same or different binging partners. The term “solid support material”, as used herein, refers to any material which is insoluble or can be made insoluble by a subsequent reaction or manipulation. Solid support materials can be latex, plastic, derivatized plastic, magnetic or non-magnetic metal, glass or silicon surface or surfaces of test tubes, microtiter wells, sheets, beads, microparticles, chips and other configurations known to those of ordinary skill in the art. To facilitate separation and/or detection of primer extension products, an extension primer can be labeled with a binding member that is specific for its binding partner which is attached to a solid material. The primer extension products can be separated from other components of the extension reaction mixture by contacting the mixture with a solid support, and then removing, from the reaction mixture, the solid support to which extension products are bound, for example, by filtration, sedimentation, washing or magnetic attraction.

For example, an allele-specific oligonucleotide can be coupled with a moiety that allows affinity capture, while other allele-specific oligonucleotides remain unmodified or are coupled with different affinity moieties. Modifications can include a sugar (for binding to a solid phase material coated with lectin), a hydrophobic group (for binding to a reverse phase column), biotin (for binding to a solid phase material coated with streptavidin), or an antigen (for binding to a solid phase material coated with an appropriate antibody). Extension reaction mixtures can be run through an affinity column, the flow-through fraction collected, and the bound fraction eluted, for example, by chemical cleavage, salt elution, and the like. Alternatively, extension reaction mixtures can be contacted with affinity capture beads.

Alternatively, each allele-specific oligonucleotide may comprise a nucleotide sequence (binding member) at its 5′ terminus, that is complementary to a nucleotide sequence (binding partner) attached to a solid support. Allele-specific oligonucleotides used in detection methods of the present invention may be coupled to an identical tag sequence (e.g., universal capture sequence) complementary to a tag probe sequence attached to a solid support. Alternatively, each allele-specific oligonucleotide may comprise a tag sequence that is allele-specific and complementary to a tag probe sequence attached to a solid support. The tag may be, for example, about 10 to about 30 nucleotides in length. Tags and specific sets of tags and tag probe sequences are disclosed, for example, in U.S. Pat. No. 6,458,530 (which is incorporated herein by reference in its entirety). In general, tag and tag sequences are selected such that they are not present in the genome (or part of the genome) of interest in order to prevent cross-hybridization with the genome. Tags are often selected in sets; and tags in a set are generally selected such that they do not cross-hybridize with another tag in the set or with the complement of another tag in the set. Tag probe sequence may be attached to multiple microparticles or to an array or micro-array. An array or micro-array may be prepared to contain a plurality of probe elements. For example, each probe elements may include a plurality of tag probes that comprise substantially the same sequence that may be of different lengths. Probe elements on an array may be arranged on the solid surface at different densities.

Methods of attaching (or immobilizing) tag sequences to a solid support are known in the art (see, for example, J. Sambrook et al., “Molecular Cloning: A Laboratory Manual”, 1989, 2^{nd} Ed., Cold Spring Harbour Laboratory Press: New York, N.Y.; “Short Protocols in Molecular Biology”, 2002, F. M. Ausubel (Ed.), 5^{th} Ed., John Wiley & Sons; U. Maskos and E. M. Southern, Nucleic Acids Res. 1992, 20: 1679-1684; R. S. Matson et al., Anal. Biochem. 1995, 224; 110-116; R. J. Lipshutz et al., Nat. Genet. 1999, 21: 20-24; Y. H. Rogers et al., Anal. Biochem. 1999, 266: 23-30; M. A. Podyminogin et al., Nucleic Acids Res. 2001, 29: 5090-5098; Y. Belosludtsev et al., Anal. Biochem. 2001, 292: 250-256; U.S. Pat. Nos. 5,427,779, 5,512,439, 5,589,586, 5,716,854 and 6,087,102). Alternatively, one can rely on commercially available systems including arrays and microarrays, such as those developed, for example, by Affymetrix, Inc. (Santa Clara, Calif.) and Illumina, Inc. (San Diego, Calif.); and multiplexed bead- and particle-based systems such as those developed by BD Biosciences (Bedford, Mass.) and Luminex, Corp. (Austin, Tex.).

After heterogeneous ASPE or after separation of extension products from other components of the ASPE reaction mixture (as described above), the presence or absence of extension products (indicative of the presence or absence of particular SNPs in the DNA sample under investigation) can be detected using any of a wide variety of methods, including spectroscopic, photochemical, biochemical, immunochemical, electrical, optical, radiochemical, and chemical methods. Selection of a method of detection will generally depend on several factors including, but not limited to, the type of assay carried out (e.g., single-plex vs. multiplex), the separation technique used, the presence or absence of a label (i.e., detectable moiety) on the extension products, and the nature of the labels (e.g., directly vs. indirectly detectable), if present.

Primer extension products generated by methods of the present invention may be detected through hybridization. For example, the extension products may be contacted with labeled nucleic acid probes. For example, each nucleic acid probe may be specific for an extension product (indicative of one allele of a SNP of interest) and may be labeled with a detectable moiety that is different from the detectable moieties carried by the other nucleic acid probes used in the assay, thereby allowing multiplex SNP detection.

Primer extension products bound to microparticles (also called microbeads) can be detected using different methods. For example, in multiplexed assays of the present invention, extension products can be simultaneously detected using pre-coded microbeads. Beads may be pre-coded using specific bead sizes, different colors and/or color intensities, different fluorescent dyes or fluorescent dye combinations.

Color-coded microspheres can be made using any of a variety of methods such as those disclosed in U.S. Pat. Nos. 6,649,414; 6,514,295; 5,073,498; 5,194,300; 5,356,713; 4,259,313; 4,283,382 and the references cited in these patents. Color-coded microspheres are also commercially available, for example, from Cortex Biochem., Inc. (San Leandro, Calif.); Seradyn, Inc. (Indianapolis, Ind.); Dynal Biotech, LLC (Brown Deer, Wis.); Spherotech, Inc. (Libertyville, Ill.); Bangs Laboratories, Inc. (Fishers, Ind.); and Polysciences, Inc. (Warrington, Pa.).

For example, polystyrene microspheres are provided by Luminex Corp. (Austin, Tex.) that are internally dyed with two spectrally distinct fluorescent dyes (x-MAP™ microbeads). Using precise ratios of these fluorophores, a large number of different fluorescent bead sets can be produced (e.g., 100 sets). Each set of beads can be distinguished by its code (or spectral signature), a combination of which allows for detection of a large number of different extension products in a single reaction vessel. The magnitude of the biomolecular interaction that occurs at the microsphere surface is measured using a third fluorochrome that acts as a reporter. These sets of fluorescent beads with distinguishable codes can be used to label extension products. Labeling (or attachment) of extension products to beads can be by any suitable means including, but not limited to, chemical or affinity capture, cross-linking, electrostatic attachment, and the like. In certain embodiments, labeling is carried out through hybridization of allele-specific tag and tag probe sequences, as described above. Because each of the different extension products is uniquely labeled with a fluorescent bead, the captured extension product (indicative of one allele of a SNP of interest) will be distinguishable from other different extension products (including extension products indicative of other alleles of the same SNP and extension products indicative of other SNPs of interest). Following tag/tag probe hybridization, the microbeads can be analyzed using different methods such as, for example, flow cytometry-based methods.

Flow cytometry is a sensitive and quantitative technique that analyzes particles in a fluid medium based on the particles' optical characteristic (H. M. Shapiro, “Practical Flow Cytometry”, 3^{rd} Ed., 1995, Alan R. Liss, Inc.; and “Flow Cytometry and Sorting, Second Edition”, Melamed et al. (Eds), 1990, Wiley-Liss: New York). A flow cytometer hydrodynamically focuses a fluid suspension of particles containing one or more fluorophores, into a thin stream so that the particles flow down the stream in a substantially single file and pass through an examination or analysis zone. A focused light beam, such as a laser beam, illustrates the particles as they flow through the examination zone, and optical detectors measure certain characteristics of the light as it interacts with the particles (e.g., light scatter and particle fluorescence at one or more wavelengths). In the stream, the microbeads are interrogated individually as they pass the detector and high-speed digital signal processing classifies each bead based on its code and quantifies the reaction on the bead surface. Thousands of beads can be interrogated per second, resulting in a high-speed, high-throughput and accurate detection of multiple different SNPs. In embodiments where the extension reaction is carried out in the presence of biotinylated dNTPs, the reaction between beads and extension products may be quantified by fluorescence after reaction with fluorescently-labeled streptavidin (e.g., Cy5-streptavidin conjugate). Instruments for performing such assay analyses are commercially available, for example, from Luminex (e.g., Luminex® 100™ Total System, Luminex® 100™ IS Total System, Luminex® High Throughput Screening System).

Extension products bound to arrays, micro-arrays or chips can be detected using different methods. In certain embodiments, primer extension products are captured (or attached) via hybridization to probes on array sites (as mentioned above). This attachment is generally a direct hybridization between an adapter sequence on the primer extension product (e.g., an allele-specific tag sequence) and a corresponding capture probe (e.g., complementary tag probe sequence) immobilized onto the surface of the array. Alternatively, the attachment can rely on indirect “sandwich” complexes using capture extender probes as known in the art (see, for example, M. Ranki et al., Gene, 1983, 21: 77-85; B. J. Connor et al., Proc. Natl. Acad. Sci. USA, 1983, 80: 278-282; and U.S. Pat. Nos. 4,563,419 and 4,751,177). The presence or absence of a bound extension product at a given spot (or position) on the array is generally determined by detecting a signal (e.g., fluorescence) from the label coupled to the product. Furthermore, since the sequence of the capture probe at each position on the array is known, the identity of an extension product at that position can be determined.

Extension products bound to arrays are often (directly or indirectly) fluorescently labeled. Methods for the detection of fluorescent labels in array configurations are known in the art and include the use of “array reading” or “scanning” systems, such as charge-coupled devices (i.e., CCDs). Any known device or method, or variation thereof can be used or adapted to practice the methods of the invention (see, for example, Y. Hiraoka et al., Science, 1987, 238: 36-41; R. S. Aikens et al., Meth. Cell Biol. 1989, 29: 291-313; A. Divane et al., Prenat. Diagn. 1994, 14: 1061-1069; S. M. Jalal et al., Mayo Clin. Proc. 1998, 73: 132-137; V. G. Cheung et al., Nature Genet. 1999, 21: 15-19; see also, for example, U.S. Pat. Nos. 5,539,517; 5,790,727; 5,846,708; 5,880,473; 5,922,617; 5,943,129; 6,049,380; 6,054,279; 6,055,325; 6,066,459; 6,140,044; 6,143,495; 6,191,425; 6,252,664; 6,261,776 and 6,294,331).

Commercially available microarray scanners are typically laser-based scanning systems that can acquire one (or more than one) fluorescent image (such as, for example, the instruments commercially available from PerkinElmer Life and Analytical Sciences, Inc. (Boston, Mass.), Virtek Vision, Inc. (Ontario, Canada) and Axon Instruments, Inc. (Union City, Calif.)). Arrays can be scanned using different laser intensities in order to ensure the detection of weak fluorescence signals and the linearity of the signal response at each spot on the array. Fluorochrome-specific optical filters may be used during acquisition of the fluorescent images. Filter sets are commercially available, for example, from Chroma Technology Corp. (Rockingham, Vt.).

A computer-assisted image analysis system is generally used to analyze fluorescent images acquired from arrays. Such systems allow for an accurate quantitation of the intensity differences and for an easy interpretation of the results. A software for fluorescence quantitation and fluorescence ratio determination at discrete spots on an array is usually included with the scanner hardware. Softwares and/or hardwares are commercially available and may be obtained from, for example, Affymetrix, Inc. (Santa Clara, Calif.), Applied Spectral Imaging, Inc. (Carlsbad, Calif.), Chroma Technology Corp. (Rockingham, Vt.), Leica Microsystems (Bannockburn, Ill.), and Vysis, Inc. (Downers Grove, Ill.).

Alternatively, a planar waveguide (PWG) chip technique can be used to detect surface bound fluorescently-labeled extension products. A waveguide refers to a two dimensional total internal reflection (TIR) element that provides an interface capable of internal reference at multiple points, thereby creating an evanescent wave that is substantially uniform across all or nearly all the entire surface. The waveguide can be comprised of transparent material such as glass, quartz, plastics such as polycarbonate, acrylic or polystyrene. The glass or other types of surfaces used for waveguides can be modified with any of a variety of functional groups including binding members such as haptens or oligonucleotide sequences (e.g., tag probe sequences).

In PWG, fluorescent excitation is carried out using an exponentially decaying evanescent light field, which preferentially excites labeled molecules that are captured within the field. Since molecules in solution (i.e., non surface bound) are not within the evanescent field, they do not get excited. This technique presents several advantages including very low fluorescent background, high dynamic range, and allows measurements in turbid solutions or optically dense suspensions. Multiplexed detection can be achieved by combining 2D arrays of ligands and CCD camera detection.

### Detection of CYP2D6 Duplication and CYP2D6 Deletion

Amplicon C which is associated with CYP2D6 deletion and Amplicon D which is associated with CYP2D6 duplication can be detected using detection probes comprising SEQ ID NOs. 85 and 86, and detection probes comprising SEQ ID NOs. 87 through 89, respectively, employing any of a variety of well-known homogeneous or heterogeneous methodologies.

Homogeneous detection methods include, but are not limited to, the use of FRET labels attached to the probes, that emit a signal in the presence of the target sequence. Molecular Beacons (S. Tyagi and F. R. Kramer, Nature Biotechnol. 1996, 14: 303-308; S. Tyagi et al., Nature Biotechnol. 1998, 16: 49-53; L. G. Kostrikis et al., Science, 1998, 279: 1228-1229; D. L. Sokol et al., Proc. Natl. Acad. Sci. USA, 1998, 95: 11538-11543; S. A. Marras et al., Genet. Anal. 1999, 14: 151-156; and U.S. Pat. Nos. 5,846,726, 5,925,517, 6,277,581 and 6,235,504) and so-called Taq-Man™ assays (U.S. Pat. Nos. 5,210,015; 5,804,375; 5487,792 and 6214,979 and WO 01/86001). Using these detection techniques, Amplicon C and Amplicon D can be detected as they are formed or in a so-called real time manner.

Other examples of homogeneous detection methods include hybridization protection assays (HPA). In such assays, the probes are labeled with acridinium ester (AE), a highly chemiluminescent molecule (Weeks et al., Clin. Chem., 1983, 29: 1474-1479; Berry et al., Clin. Chem., 1988, 34: 2087-2090), using a non-nucleotide-based linker arm chemistry (U.S. Pat. Nos. 5,585,481 and 5,185,439). Chemiluminescence is triggered by AE hydrolysis with alkaline hydrogen peroxide, which yields an excited N-methyl acridone that subsequently deactivates with emission of a photon. In the absence of a target sequence, AE hydrolysis is rapid. However, the rate of AE hydrolysis if greatly reduced when the probe is bound to the target sequence. Thus, hybridized and un-hybridized AE-labeled probes can be detected directly in solution, without the need for physical separation.

Heterogeneous detection systems are well-known in the art and generally employ a capture agent to separate amplified sequences from other materials in the reaction mixture. Capture agents typically comprise a solid support material (e.g., microtiter wells, beads, chips, and the like) coated with one or more specific binding sequences. A binding sequence may be complementary to a tail sequence added to the oligonucleotide probes of the invention. Alternatively, a binding sequence may be complementary to a sequence of a capture oligonucleotide, itself comprising a sequence complementary to a tail sequence of an inventive oligonucleotide probe. After separation of the amplification product/probe hybrids bound to the capture agents from the remaining reaction mixture, the amplification product/probe hybrids can be detected using any detection methods described above.

### Controls

In certain embodiments of the invention, an internal control or an internal standard is added to the biological sample (or to purified/isolated nucleic acid extracted from the biological sample) to serve as a control for extraction and/or target amplification. Preferably, the internal control includes a sequence that differs from the target sequence(s), and is capable of amplification by the primers used to amplify the target sequence(s). The use of an internal control allows for the monitoring of the extraction process, amplification reaction, and detection, and control of the assay performance. The amplified control and amplified target(s) are typically distinguished at the detection step by using different probes (e.g., labeled with different detectable agents) for the detection of the control and target. As will be appreciated by one of ordinary skill in the art, more than one internal control can be used.

### Multiplex Detection of CYP2D6 Polymorphisms

In certain embodiments, the methods of the present invention are used to determine the genotype of an individual with respect to both CYP2D6 alleles present in that individual's genome. In some embodiments, the methods of the present invention are used to detect the presence of multiple polymorphic variants (e.g., polymorphic variants at a plurality of polymorphic sites) in parallel or otherwise substantially simultaneously.

Oligonucleotide arrays represent one suitable means for doing so. Methods can also be used in which detection probes are attached to microparticles or are modified to be capable of attachment to microparticles (as described above). Other methods, including methods in which reactions (e.g., amplification, detection) are performed in individual vessels (e.g., within individual wells of a multi-well plate or other vessel) may also be performed so as to detect the presence of multiple polymorphic variants (e.g., polymorphic variants at a plurality of polymorphic sites) in parallel or substantially simultaneously.

Using such methods, the presence or absence of a plurality of polymorphic variants at different polymorphic sites can be detected. Thus, a genetic profile for an individual is generated, wherein the genetic profile indicates which allelic variant is present at a plurality of different polymorphisms that are associated with adverse drug reaction.

### III—Uses of Inventive Oligonucleotide Sequences and Detection Methods

The invention provides a variety of methods for determining the identity of CYP2D6 allele(s) present in an individual. The inventive methods can be used, for example, to predict how such an individual will respond to drugs or other xenobiotic compounds that are metabolized, at least in part, by CYP2D6.

As but one limiting example, an individual that carries one or more “defective” CYP2D6 alleles, which defective alleles do not function to metabolize one or more particular drugs, may be susceptible to toxicity and/or to an otherwise adverse drug reaction since such an individual will be unable to metabolize the drugs as quickly as an individual carrying one or more normal CYP2D6 alleles, and the active, non-metabolized drug will remain in the individual's system for a longer period of time.

Thus, determining that an individual carries on or more such defective CYP2D6 alleles can be used to predict whether such an individual is susceptible to toxicity and/or to an otherwise adverse drug reaction. In certain embodiments, determining that an individual carries one or more such defective CYP2D6 alleles can be used to select an appropriate therapeutic regimen including, but not limited to, selecting one or more appropriate drugs, modulating drug dose, modulating dosing interval, etc. In certain embodiments, an individual that carries one or more such defective CYP2D6 alleles can be administered a different drug or other therapeutic regimen, such that any potential toxicity is avoided altogether.

Similarly, an individual that carries one or more “hyperactive” CYP2D6 alleles, which hyperactive alleles function by metabolizing one or more particular drugs more quickly or otherwise more effectively, may be completely or partially immunity to a therapeutic regimen based on one or more particular drugs metabolized by CYP2D6 since such an individual will metabolize the drug more quickly than an individual with one or more normal CYP2D6 alleles, and the active drug will therefore be cleared from the individual's system more quickly. In certain embodiments, determining that an individual carried one or more such hyperactive CYP2D6 alleles can be used to select an appropriate therapeutic regimen including, but not limited to, selecting one or more appropriate drugs, modulating drug dose, modulating dosing interval, etc. In certain embodiments, an individual that carries one or more such hyperactive CYP2D6 alleles can be administered a different drug or other therapeutic regimen, such that that individual will advantageously respond to the drug or therapeutic regimen that is administered.

In certain embodiments, a drug is administered to an individual in a pro-drug form in which the administered drug itself exhibits little or no activity. However, such a drug may be subject to metabolization by CYP2D6 such that upon metabolization, an active metabolic product is generated. In such embodiments, an individual carrying one or more “defective” CYP2D6 alleles may exhibit complete or partial immunity to a therapeutic regimen based on that drug since less metabolic product, or no metabolic product, will be generated. Similarly, an individual carrying one or more “hyperactive” CYP2D6 alleles may exhibit susceptibility to toxicity and/or to an otherwise adverse drug reaction since such an individual will metabolize more of the inactive pro-drug (or will metabolize the pro-drug more quickly) than an individual carrying one or more normal CYP2D6 alleles.

In certain embodiments of the present invention, a panel of CYP2D6 polymorphisms (e.g., two or more SNPs) is defined that provides diagnostic and/or prognostic information when an individual is genotyped with respect to the SNPs. In certain embodiments, results obtained from the panel predict the risk of developing adverse drug response. The risk can be, e.g., absolute risk, which can be expressed in terms of likelihood (e.g., % likelihood) that an individual will experience adverse drug response. The risk can be expressed in terms of relative risk, e.g., a factor that expresses the degree to which the individual is at increased risk relative to the risk the individual would face if his or her genotype with respect to one or more of the polymorphisms was different. Individuals can be stratified based on their risk. Such stratification can be used, for example, to select individuals who would be likely to benefit from particular therapeutic regimens. It should be emphasized that the information provided by the methods of the present invention can be qualitative or quantitative and can be expressed using any convenient means.

It will be appreciated by one skilled in the art that the risk obtained using methods according to the present invention may be compared to and/or combined with results from other tests or assays performed for determining the susceptibility of an individual to toxicity and/or otherwise adverse drug reaction. Such comparison and/or combination may help to guide specific and individualized therapy, e.g., to optimize treatment and avoid drug adverse response.

### IV—Kits

In another aspect, the present invention provides kits comprising materials useful for the detection and identification of CYP2D6 polymorphisms according to methods described herein. The inventive kits may be used by diagnostic laboratories, experimental laboratories, or practitioners. The invention provides kits which can be used in these different settings.

Materials and reagents useful for the detection of CYP2D6 polymorphisms according to the present invention may be assembled together in a kit. In certain embodiments, an inventive kit comprises at least one inventive primer set and/or primer/probe set, and optionally, amplification reaction reagents and/or amplification reaction reagents and primer extension reagents. Each kit necessarily comprises the reagents which render the procedure specific. Thus a kit intended to be used for the detection of a particular SNP preferably comprises oligonucleotide sequences described herein that can be used to amplify a CYP2D6 target sequence that comprises the particular SNP and oligonucleotide sequences described herein that can be used in ASPE for detecting the SNP of interest. A kit intended to be used for the multiplex detection of a plurality of SNPs preferably comprises a plurality of oligonucleotide sequences described herein that can be used to amplify CYP2D6 target sequences that comprise the SNPs and oligonucleotide sequences described herein that can be used in ASPE reactions to detect the SNPs of interest.

Suitable amplification/primer extension reaction reagents include, for example, one or more of: buffers; enzymes having reverse transcriptase and/or polymerase activity or exonuclease activity; enzymes having polymerase activity and lacking 5′→3′ exonuclease activity or both 5′→3′ and 3′→5′ exonuclease activity; enzyme cofactors such as magnesium or manganese; salts; nicotinamide adenide dinuclease (NAD); and deoxynucleoside triphosphates (dNTPs) such as, for example, deoxyadenosine triphospate; deoxyguanosine triphosphate, deoxycytidine triphosphate and thymidine triphosphate, biotinylated dNTPs, suitable for carrying out the amplification/ASPE reactions.

Depending on the procedure, an inventive kit may further comprise one or more of: wash buffers and/or reagents; hybridization buffers and/or reagents; labeling buffers and/or reagents; and detection means. Buffers and/or reagents are preferably optimized for the particular amplification/detection technique for which the kit is intended. Protocols for using these buffers and reagents for performing different steps of the procedure may also be included in the kit.

Furthermore, a kit may be provided with an internal control as a check on the amplification procedure and to prevent occurrence of false negative test results due to failures in the amplification procedure. An optimal control sequence is selected in such a way that it will not compete with the target nucleic acid sequence(s) in the amplification reaction (as described above).

Kits may also contain reagents for the isolation of nucleic acids from biological samples prior to amplification and/or reagents for the separation/purification of amplified CYP2D6 target sequence(s) of interest.

Reagents may be supplied in a solid (e.g., lyophilized) or liquid form. The kits of the present invention optionally comprise different containers (e.g., vial, ampoule, test tube, flash, or bottle) for each individual buffer and/or reagent. Each component will generally be suitable as aliquoted in its respective container or provided in a concentrated form. Other containers suitable for conducting certain steps of the amplification/detection assay may also be provided. The individual containers of the kit are preferably maintained in close confinement for commercial use.

In embodiments where the kit comprises primers and/or probes suitable for detection of a plurality of CYP2D6 polymorphic variants, the probes may be covalently or non-covalently attached to microparticles (e.g., beads). Alternatively, the probes may be covalently or non-covalently attached to a substantially planar, rigid substrate or support. The substrate may be transparent to radiation of the excitation and emission wavelengths used for excitation and detection of typical labels (e.g., fluorescent labels, quantum dots, plasmon resonant particles, nanoclusters), e.g., 400 to 900 nm. Materials such as glass, plastic, quartz, etc. are suitable. For example, a glass slide or the like can be used.

In certain embodiments, the kits of the invention are adaptable to high-throughput and/or automated operation. For example, the kits may be suitable for performing assays in multi-well plates and may utilize automated fluid handling and/or robotic systems, plate readers, etc. In some embodiments, flow cytometry is used.

One of ordinary skill in the art will appreciate that a number of other polymorphisms associated with adverse drug response are known in the art, including other CYP2D6 polymorphisms as well as polymorphisms of other cytochrome P540 genes. In certain embodiments, oligonucleotide sequences for amplification primers and/or detection probes specific for other CYP2D6 polymorphisms and/or polymorphisms of other cytochrome P540 genes (e.g., CYP2C9) associated with adverse drug response are included in the inventive kit. For example, at least 50%, at least 60%, at least 70%, at least 80%, at least 90% or more of the primers and/or probes in the kits comprise CYP2D6-specific oligonucleotide sequences described herein.

An inventive kit may also comprise instructions for using the amplification/ASPE reaction reagents and primer sets or primer/probe sets according to the present invention. Instructions for using the kit according to one or more methods of the invention may comprise instructions for processing the biological sample, extracting nucleic acid molecules from the sample, and/or performing the test; instructions for interpreting the results, including for using the results for diagnosis of an individual at risk for adverse drug response. For example, the kit may comprise an informational sheet or the like that describes how to interpret the results of the test and/or how to utilize the results of the test together with information regarding the existence or value of one or more classical risk factors in the individual. The kit may also comprise a notice in the form prescribed by a government agency (e.g., FDA) regulating the manufacture, use or sale of pharmaceuticals of biological products. An identifier, e.g., a bar code, radio frequency, ID tag, etc., may be present in or on the kit. The identifier can be used, e.g., to uniquely identify the kit for purposes of quality control, inventory control, tracking, movement between workstations, etc. According to certain embodiments of the invention, the kits are manufactured in accordance with good manufacturing practices as required for FDA-approved diagnostic kits.

### V—Computer-Readable Media

The invention further provides a database or other suitably organized and optionally searchable compendium of information stored on a computer-readable medium and comprising results obtained by performing one or more of the methods of the invention on one or more samples (e.g., on a plurality of samples obtained from a plurality of individuals).

The computer-readable medium can be any form of storage medium such as a computer hard disc, compact disc, zip disc, magnetic tape, flash memory, etc. It will be appreciated that the information can be stored in a wide variety of formats. The database may include results of genotyping one or more individuals with respect to one or more of the CYP2D6 polymorphisms described herein. The results can be presented in any of a wide variety of formats, provided that the information allows one of ordinary skill in the art to discern that at least one, and advantageously more than one, CYP2D6 polymorphism is present in the individual. In certain embodiments of the invention, the information allows one of ordinary skill in the art to determine whether the individual possesses a CYP2D6 polymorphism selected from the group consisting of CYP2D6 duplication, CYP2D6 deletion (*5) and the following 12 SNPs of CYP2D6: −1584 C>G (*2A); 124 G>A (*12); 100 C>T (*4, *10); 883 G>C (*11); 1023 C>T (*17); 1707 T>del (*6); 1758 G>T (*8); 1846 G>A (*4); 2549 A>del (*3); 2613-1615 del AGA (*9); 2850 C>T (*2, *17); and 2935 A>C (*7). In certain embodiments, the information allows one of ordinary skill in the art to determine the identity of each of two CYP2D6 alleles present in the individual. The invention also encompasses a method comprising the step of electronically sending or receiving information such as that present in a database of the invention and/or electronically sending or receiving results of a genotyping test as described herein.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

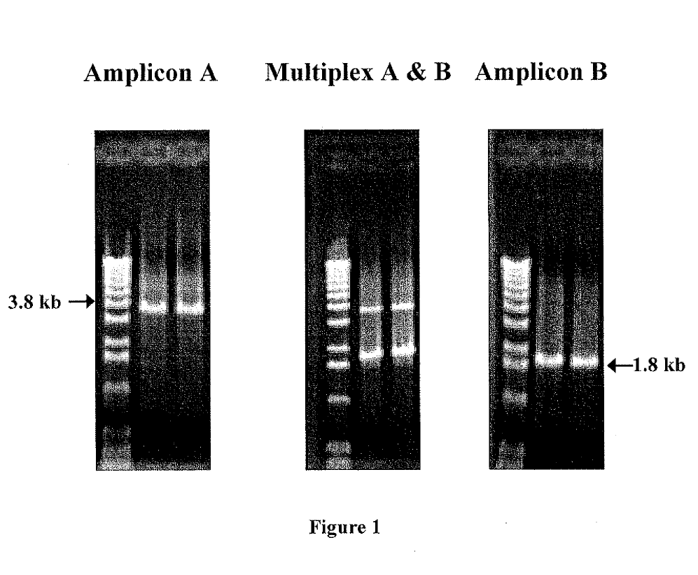

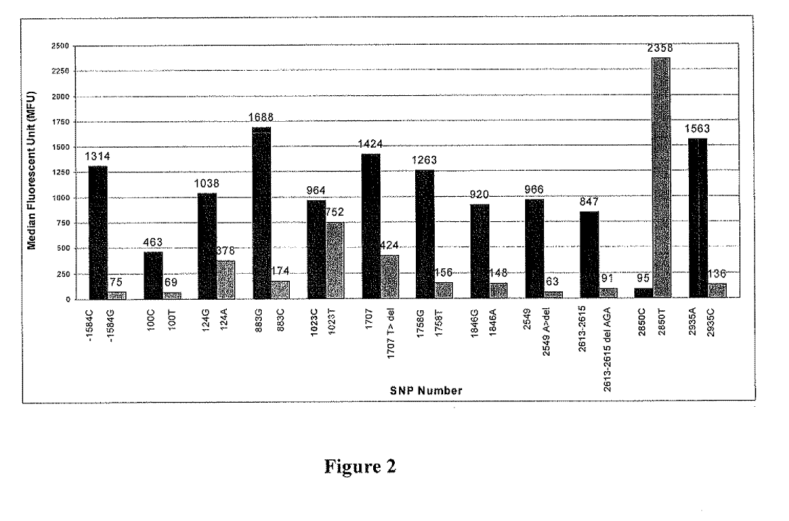

An assay was carried out in a multiplex format using two sets of primers and detection probes described in Table 1 (SEQ ID NOs. 1, 2, 3 and 4) and Table 2 (SEQ ID NOs. 11 through 83). Amplification of genomic DNA was followed by the Allele Specific Primer Extension (ASPE) reaction. ASPE capture on Luminex beads showed good discrimination of all of the 12 SNPs described herein, as shown on FIG. 1 and FIG. 2.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

TABLE 1

| SEQ ID | Sequence | | |
|---|---|---|---|
| NO. | Name* | Sequence (5′ → 3′) | Strand |
| 1 | 5173U20 | CGCCGTCAAGCTTTCCGACA | (+) |
| 2 | 8934L20 | CGCCCTCGTCCCCATGCTCA | (-) |
| 3 | 2922U21 | CAGCTGGAATCCGGTGTCGAA | (+) |
| 4 | 4710L21 | CGGCCCTGACACTCCTTCTTG | (-) |
| 5 | 138U18 | GCAGGGAGCCCACCGTAG | (+) |
| 6 | 423U20 | CGCCTCTCCCTCCATACCTC | (+) |
| 7 | 3528L22 | TACAGGCATGAGCTAAGGCACC | (-) |
| 8 | 3534L23 | CTGGGATTACAGGCATGAGCTAA | (-) |
| 9 | 6308U21 | ACGGAAGACAAATCATGGCGT | (+) |
| 10 | 9519L21 | TCAACTTTCCCTTAGCCGTCA | (-) |

*“U” stands for upper or forward primer complementary to the coding or sense strand (+), and “L”+10 stands for lower or reverse primer complementary to the coding or sense strand (-).

TABLE 2

| SEQ ID | Sequence | | | |
|---|---|---|---|---|
| NO. | Name* | Sequence (5′ → 3′) | Strand | Detection |
| 11 | #3W | CCTGGACAACTTGGAAGAACCC | (+) | -1584 C > G |
| 12 | #3M | CCTGGACAACTTGGAAGAACCG | (+) | |
| 13 | #4W | GCTGGGCTGCACGCTACC | (+) | 100 C > T |
| 14 | #4M | GCTGGGCTGCACGCTACT | (+) | |
| 15 | #4W + 1 | CGCTGGGCTGCACGCTACC | (+) | |
| 16 | #4M + 1 | CGCTGGGCTGCACGCTACT | (+) | |
| 17 | #4W - 1 | CTGGGCTGCACGCTACC | (+) | |
| 18 | #4M - 1 | CTGGGCTGCACGCTACT | (+) | |
| 19 | #4(-)W | CAGGGGGCCTGGTGG | (-) | |
| 20 | #4(-)M | GCAGGGGGCCTGGTGA | (-) | |
| 21 | #4(-)W + 1 | GCAGGGGGCCTGGTGG | (-) | |
| 22 | #4(-)M + 1 | GGCAGGGGGCCTGGTGA | (-) | |
| 23 | #4(-)W - 1 | AGGGGGCCTGGTGG | (-) | |
| 24 | #4(-)M - 1 | CAGGGGGCCTGGTGA | (-) | |
| 25 | #5W | CCCTGCCACTGCCCG | (+) | 124 G > A |
| 26 | #5M | CCCCTGCCACTGCCCA | (+) | |
| 27 | #5W + 1 | CCCCTGCCACTGCCCG | (+) | |
| 28 | #5M + 1 | CCCCCTGCCACTGCCCA | (+) | |
| 29 | #5W - 1 | CCTGCCACTGCCCG | (+) | |
| 30 | #5M - 1 | CCCTGCCACTGCCCA | (+) | |
| 31 | #5(-)W | AGCAGGTTGCCCAGCCC | (-) | |
| 32 | #5(-)M | CAGCAGGTTGCCCAGCCT | (-) | |
| 33 | #6W | CCTGACCCTCCCTCTGCAG | (+) | 833 G > C |
| 34 | #6M | CCTGACCCTCCCTCTGCAC | (+) | |
| 35 | #6(-)W | AGCGGCGCCGCAAC | (-) | |
| 36 | #6(-)M | AGCGGCGCCGCAAG | (-) | |
| 37 | #7W | CCGCCTGTGCCCATCAC | (+) | 1023 C > T |
| 38 | #7M | CCGCCTGTGCCCATCAT | (+) | |
| 39 | #7W + 1 | CCCGCCTGTGCCCATCAC | (+) | |
| 40 | #7M + 1 | CCCGCCTGTGCCCATCAT | (+) | |
| 41 | #7W - 1 | CGCCTGTGCCCATCAC | (+) | |
| 42 | #7M - 1 | CGCCTGTGCCCATCAT | (+) | |
| 43 | #7(-)W | CGAAACCCAGGATCTGGG | (-) | |
| 44 | #7(-)M | CCGAAACCCAGGATCTGGA | (-) | |
| 45 | #8W | GCAAGAAGTCGCTGGAGCAGT | (+) | 1707 T > del |
| 46 | #8M | CAAGAAGTCGCTGGAGCAGG | (+) | |
| 47 | #8W + 1 | GGCAAGAAGTCGCTGGAGCAGT | (+) | |
| 48 | #8M + 1 | GCAAGAAGTCGCTGGAGCAGG | (+) | |
| 49 | #8W - 1 | CAAGAAGTCGCTGGAGCAGT | (+) | |
| 50 | #8M - 1 | AAGAAGTCGCTGGAGCAGG | (+) | |
| 51 | #8W(-) | GCCTCCTCGGTCACCCA | (-) | |
| 52 | #8M(-) | GCCTCCTCGGTCACCCC | (-) | |
| 53 | #9W | CCTTCGCCAACCACTCCG | (+) | 1758 G > T |
| 54 | #9M | GCCTTCGCCAACCACTCCT | (+) | |
| 55 | #9W(-) | CTTCTGCCCATCACCCACC | (-) | |
| 56 | #9M(-) | CTTCTGCCCATCACCCACA | (-) | |
| 57 | #10W | GCATCTCCCACCCCCAG | (+) | 1846 G > A |
| 58 | #10M | GCATCTCCCACCCCCAA | (+) | |
| 59 | #10W + 1 | CGCATCTCCCACCCCCAG | (+) | |
| 60 | #10M + 1 | CGCATCTCCCACCCCCAA | (+) | |
| 61 | #10W - 1 | CATCTCCCACCCCCAG | (+) | |
| 62 | #10M - 1 | CATCTCCCACCCCCAA | (+) | |
| 63 | #10(-)W | GGCGAAAGGGGCGTCC | (-) | |
| 64 | #10(-)W - 1 | GCGAAAGGGGCGTCC | (-) | |
| 65 | #10(-)M | GGCGAAAGGGGCGTCT | (-) | |
| 66 | #11W | GGATGAGCTGCTAACTGAGCACA | (+) | 2549 A > del |
| 67 | #11M | GATGAGCTGCTAACTGAGCACG | (+) | |
| 68 | #12W | CCTTCCTGGCAGAGATGGAGA | (+) | 2613-2615 |
| 69 | #12M | CTTCCTGGCAGAGATGGAGGT | (+) | del AGA |
| 70 | #12W + 1 | GCCTTCCTGGCAGAGATGGAGA | (+) | |
| 71 | #12M + 1(3′) | CTTCCTGGCAGAGATGGAGGT | (+) | |
| 72 | #12M + 2(3′ & 5′) | CCTTCCTGGCAGAGATGGAGGT | (+) | |
| 73 | #12M + 1 (5′) | CCTTCCTGGCAGAGATGGAGG | (+) | |
| 74 | #12W - 1 | CTTCCTGGCAGAGATGGAGA | (+) | |
| 75 | #12M - 1 | CTTCCTGGCAGAGATGGAGG | (+) | |
| 76 | #13W + 1 | GCAGCTTCAATGATGAGAACCTGC | (+) | 2850 C > T |
| 77 | #13M + 1 | AGCAGCTTCAATGTGAGAACCTGT | (+) | |
| 78 | #13W | CAGCTTCAATGATGAGAACCTGC | (+) | |
| 79 | #13M | GCAGCTTCAATGATGAGAACCTGT | (+) | |
| 80 | #13W - 1 | AGCTTCAATGATGAGAACCTGC | (+) | |
| 81 | #13M - 1 | AGCTTCAATGATGAGAACCTGT | (+) | |
| 82 | #14W | GCCTCCTGCTCATGATCCTACA | (+) | 2935 A > C |
| 83 | #14M | GCCTCCTGCTCATGATCCTACC | (+) | |
| 84 | Del-1 | GGAGGCGCTGAGAGCGA | (+) | Gene |
| 85 | Del-2 | CCATACCTCCCCGCAAGC | (+) | deletion |
| 86 | Dup-1 | CCTCAGGGATGCTGCTGTACA | (+) | Gene |
| 87 | Dup-2 | GCAGTGAGCCGAGATCACAC | (+) | duplication |
| 88 | Dup-3 | TGCACTCCAGTCTGGGTGATAAGTA | (+) | |

*“W” stands for wild-type probe, and “M”+10 stands for mutant probe.

1. An isolated oligonucleotide comprising a nucleic acid sequence selected from the group consisting of SEQ ID NOs. 1-88, complementary sequences thereof, active fragments thereof, and combinations thereof.

2. An isolated oligonucleotide according to claim 1 wherein the oligonucleotide is an amplification primer comprising a nucleic acid sequence selected from the group consisting of SEQ. ID NOs. 1-10, complementary sequences thereof, active fragments thereof, and combinations thereof.

3. The isolated oligonucleotide amplification primer of claim 2 having a nucleic acid sequence selected from the group consisting of SEQ. ID NOs. 1-10.

4. An isolated oligonucleotide according to claim 1 wherein the oligonucleotide is a detection probe.

5. The isolated oligonucleotide detection probe of claim 4 having a nucleic acid sequence selected from the group consisting of SEQ. ID NOs. 11-88.

6. A primer pair for amplifying a portion of a CYP2D6 gene or a portion of genomic DNA comprising a CYP2D6 deletion or duplication by PCR, wherein the primer pair is selected from the group consisting of:
(a) Primer Pair 1 comprising a forward primer comprising SEQ. ID NO. 1 or any active fragment thereof, and a reverse primer comprising SEQ ID NO. 2 or any active fragment thereof;
(b) Primer Pair 2 comprising a forward primer comprising SEQ ID NO. 3 or any active fragment thereof, and a reverse primer comprising SEQ ID NO. 4 or any active fragment thereof;
(c) Primer Pair 3 comprising a forward primer comprising SEQ ID NO. 5 or any active fragment thereof, and a reverse primer comprising SEQ ID NO. 7 or any active fragment thereof;
(d) Primer Pair 4 comprising a forward primer comprising SEQ ID NO. 6 or any active fragment thereof, and a reverse primer comprising SEQ ID NO. 7 or any active fragment thereof;
(e) Primer Pair 5 comprising a forward primer comprising SEQ ID NO. 6 or any active fragment thereof, and a reverse primer comprising SEQ ID NO. 8 or any active fragment thereof; and
(f) Primer Pair 6 comprising a forward primer comprising SEQ ID NO. 9 or any active fragment thereof, and a reverse primer comprising SEQ ID NO. 10 or any active fragment thereof.

7. A pair of allele-specific extension probes which can distinguish between CYP2D6 alleles that differ at a polymorphic position when used in a primer extension assay, wherein one of said extension probes is complementary to a wild-type CYP2D6 allele at the polymorphic position and the other of said extension probes is complementary to a mutant CYP2D6 allele at the polymorphic position, wherein said polymorphic position is selected from the group consisting of: nucleotide −1584, nucleotide 100, nucleotide 124, nucleotide 833, nucleotide 1023, nucleotide 1707, nucleotide 1758, nucleotide 1846, nucleotide 2549, nucleotides 2613-2615, nucleotide 2850 and nucleotide 2935.

8. The pair of allele-specific extension probes of claim 7, wherein the pair is selected from the group consisting of:
a probe pair comprising a wild-type probe and a mutant probe comprising sequences selected from the group consisting of SEQ ID NOs 11 and 12 and any active fragment thereof;
a probe pair comprising a wild-type probe and a mutant probe comprising sequences selected from the group consisting of SEQ ID NOs. 13-24 and any active fragment thereof;
a probe pair comprising a wild-type probe and a mutant probe comprising sequences selected from the group consisting of SEQ ID NOs. 25-32 and any active fragment thereof;
a probe pair comprising a wild-type probe and a mutant probe comprising sequences selected from the group consisting of SEQ ID NOs. 33-36 and any active fragment thereof;
a probe pair comprising a wild-type probe and a mutant probe comprising sequences selected from the group consisting of SEQ ID NOs. 37-44 and any active fragment thereof;
a probe pair comprising a wild-type probe and a mutant probe comprising sequences selected from the group consisting of SEQ ID NOs. 45-52 and any active fragment thereof;
a probe pair comprising a wild-type probe and a mutant probe comprising sequences selected from the group consisting of SEQ ID NOs. 53-56 and any active fragment thereof;
a probe pair comprising a wild-type probe and a mutant probe comprising sequences selected from the group consisting of SEQ ID NOs. 57-65 and any active fragment thereof;
a probe pair comprising a wild-type probe and a mutant probe comprising sequences selected from the group consisting of SEQ ID NOs 66 and 67 and any active fragment thereof;
a probe pair comprising a wild-type probe and a mutant probe comprising sequences selected from the group consisting of SEQ ID NOs. 68-75 and any active fragment thereof;
a probe pair comprising a wild-type probe and a mutant probe comprising sequences selected from the group consisting of SEQ ID NOs. 76-81 and any active fragment thereof; and
a probe pair comprising a wild-type probe and a mutant probe comprising sequences selected from the group consisting of SEQ ID NOs. 82 and 83 and any active fragment thereof.

9. A kit comprising a collection of primer pairs, wherein said primer pairs are suitable for use in a single-plex or multiplex PCR reaction that comprises human genomic DNA, said collection comprising:
(a) a primer pair which, when used in the PCR reaction, generates an amplification product that encompasses nucleotides 5173 to 8953 of the CYP2D6 gene (Accession NG_{—}003180);
(b) a primer pair which, when used in the PCR reaction, generates an amplification product that encompasses nucleotides 2922 to 8953 of the CYP2D6 gene (Accession M_{—}33388);
(c) a primer pair which, when used in the PCR reaction, generates an amplification product only if the genomic DNA contains a CYP2D6 deletion; and
(d) a primer pair which, when used in the PCR reaction, generates and amplification product only if the genomic DNA contains a CYP2D6 duplication.

10. The kit of claim 9, wherein the primer pairs do not significantly amplify CYP2D7 or CYP2D8 sequences present in the PCR reaction.

11. The kit of claim 10, comprising the following primer pairs:
(a) Primer Pair 1 comprising a forward primer comprising SEQ. ID NO. 1 or any active fragment thereof, and a reverse primer comprising SEQ ID NO. 2 or any active fragment thereof;
(b) Primer Pair 2 comprising a forward primer comprising SEQ ID NO. 3 or any active fragment thereof, and a reverse primer comprising SEQ ID NO. 4 or any active fragment thereof;
(c) at least one primer pair selected from the group consisting of:
(i) Primer Pair 3 comprising a forward primer comprising SEQ ID NO. 5 or any active fragment thereof, and a reverse primer comprising SEQ ID NO. 7 or any active fragment thereof;
(ii) Primer Pair 4 comprising a forward primer comprising SEQ ID NO. 6 or any active fragment thereof, and a reverse primer comprising SEQ ID NO. 7 or any active fragment thereof; and
(iii) Primer Pair 5 a forward primer comprising SEQ ID NO. 6 or any active fragment thereof, and a reverse primer comprising SEQ ID NO. 8 or any active fragment thereof;
(d) Primer Pair 6 a forward primer comprising SEQ ID NO. 9 or any active fragment thereof, and a reverse primer comprising SEQ ID NO. 10 or any active fragment thereof.

12. A primer/probe set for detecting a CYP2D6 polymorphism, wherein the primer/probe set is selected from the group consisting of:
(a) Primer Pair 1 comprising a forward primer comprising SEQ. ID NO. 1 or any active fragment thereof, and a reverse primer comprising SEQ ID NO. 2 or any active fragment thereof; and at least one probe pair selected from the group consisting of:
a probe pair comprising a wild-type probe and a mutant probe comprising sequences selected from the group consisting of SEQ ID NOs 11 and 12 and any active fragment thereof;
a probe pair comprising a wild-type probe and a mutant probe comprising sequences selected from the group consisting of SEQ ID NOs. 13-24 and any active fragment thereof;
a probe pair comprising a wild-type probe and a mutant probe comprising sequences selected from the group consisting of SEQ ID NOs. 25-32 and any active fragment thereof;
a probe pair comprising a wild-type probe and a mutant probe comprising sequences selected from the group consisting of SEQ ID NOs. 33-36 and any active fragment thereof; and
a probe pair comprising a wild-type probe and a mutant probe comprising sequences selected from the group consisting of SEQ ID NOs. 37-44 and any active fragment thereof;
(b) Primer Pair 2 comprising a forward primer comprising SEQ ID NO. 3 or any active fragment thereof, and a reverse primer comprising SEQ ID NO. 4 or any active fragment thereof; and at least one probe pair selected from the group consisting of:
a probe pair comprising a wild-type probe and a mutant probe comprising sequences selected from the group consisting of SEQ ID NOs. 45-52 and any active fragment thereof;
a probe pair comprising a wild-type probe and a mutant probe comprising sequences selected from the group consisting of SEQ ID NOs. 53-56 and any active fragment thereof;
a probe pair comprising a wild-type probe and a mutant probe comprising sequences selected from the group consisting of SEQ ID NOs. 57-65 and any active fragment thereof;
a probe pair comprising a wild-type probe and a mutant probe comprising sequences selected from the group consisting of SEQ ID NOs 66 and 67 and any active fragment thereof;
a probe pair comprising a wild-type probe and a mutant probe comprising sequences selected from the group consisting of SEQ ID NOs. 68-75 and any active fragment thereof;
a probe pair comprising a wild-type probe and a mutant probe comprising sequences selected from the group consisting of SEQ ID NOs. 76-81 and any active fragment thereof; and
a probe pair comprising a wild-type probe and a mutant probe comprising sequences selected from the group consisting of SEQ ID NOs. 82 and 83 and any active fragment thereof.
(c) Primer Pair 3 comprising a forward primer comprising SEQ ID NO. 5 or any active fragment thereof, and a reverse primer comprising SEQ ID NO. 7 or any active fragment thereof; and at least one probe comprising a sequence selected from the group consisting of SEQ ID NO. 84, SEQ ID NO. 85 and any active fragment thereof;
(d) Primer Pair 4 comprising a forward primer comprising SEQ ID NO. 6 or any active fragment thereof, and a reverse primer comprising SEQ ID NO. 7 or any active fragment thereof; and at least one probe comprising a sequence selected from the group consisting of SEQ ID NO. 84, SEQ ID NO. 85 and any active fragment thereof;
(e) Primer Pair 5 a forward primer comprising SEQ ID NO. 6 or any active fragment thereof, and a reverse primer comprising SEQ ID NO. 8 or any active fragment thereof; and at least one probe comprising a sequence selected from the group consisting of SEQ ID NO. 84, SEQ ID NO. 85, and any active fragment thereof;
(f) Primer Pair 6 a forward primer comprising SEQ ID NO. 9 or any active fragment thereof, and a reverse primer comprising SEQ ID NO. 10 or any active fragment thereof; and at least one probe comprising a sequence selected from the group consisting of SEQ ID NO. 86, SEQ ID NO. 87, SEQ ID NO. 88, and any active fragment thereof.

13. A kit according to claim 9 wherein
a primer pair that, when used in the PCR reaction, generates an amplification product that encompasses nucleotides 2922 to 4730 of the CYP2D6 gene (Accession M_{—}33388).

14-15. (canceled)

16. The kit of claim 13 further comprising a collection of probes comprising:
(a′) at least one probe pair that can be used in an ASPE reaction to detect a SNP that resides within the amplification product generated by the primer pair set forth in (a);
(b′) at least one probe pair that can be used in an ASPE reaction to detect a SNP that resides within the amplification product generated by the primer pair set forth in (b);
(c′) at least one probe that hybridizes to the amplification product generated by the primer pair set forth in (c); and
(d′) at least one probe that hybridizes to the amplification product generated by the primer pair set forth in (d).

17. The kit of claim 16 further comprising a collection of probes comprising:
(a′) at least one probe pair selected from the group consisting of:
a probe pair comprising a wild-type probe and a mutant probe comprising sequences selected from the group consisting of SEQ ID NOs 11 and 12 and any active fragment thereof;
a probe pair comprising a wild-type probe and a mutant probe comprising sequences selected from the group consisting of SEQ ID NOs. 13-24 and any active fragment thereof;
a probe pair comprising a wild-type probe and a mutant probe comprising sequences selected from the group consisting of SEQ ID NOs. 25-32 and any active fragment thereof;
a probe pair comprising a wild-type probe and a mutant probe comprising sequences selected from the group consisting of SEQ ID NOs. 33-36 and any active fragment thereof; and
a probe pair comprising a wild-type probe and a mutant probe comprising sequences selected from the group consisting of SEQ ID NOs. 37-44 and any active fragment thereof;
(b′) at least one probe pair selected from the group consisting of:
a probe pair comprising a wild-type probe and a mutant probe comprising sequences selected from the group consisting of SEQ ID NOs. 45-52 and any active fragment thereof;
a probe pair comprising a wild-type probe and a mutant probe comprising sequences selected from the group consisting of SEQ ID NOs. 53-56 and any active fragment thereof;
a probe pair comprising a wild-type probe and a mutant probe comprising sequences selected from the group consisting of SEQ ID NOs. 57-65 and any active fragment thereof;
a probe pair comprising a wild-type probe and a mutant probe comprising sequences selected from the group consisting of SEQ ID NOs 66 and 67 and any active fragment thereof;
a probe pair comprising a wild-type probe and a mutant probe comprising sequences selected from the group consisting of SEQ ID NOs. 68-75 and any active fragment thereof;
a probe pair comprising a wild-type probe and a mutant probe comprising sequences selected from the group consisting of SEQ ID NOs. 76-81 and any active fragment thereof; and
a probe pair comprising a wild-type probe and a mutant probe comprising sequences selected from the group consisting of SEQ ID NOs. 82 and 83 and any active fragment thereof;
(c′) at least one probe selected from the group consisting of:
a probe comprising a sequence selected from the group consisting of SEQ ID NO. 84, SEQ ID NO. 85 any active fragment thereof; and
(d′) at least one probe selected from the group consisting of:
a probe comprising a sequence selected from the group consisting of SEQ ID NO. 86, SEQ ID NO. 87, SEQ ID NO. 88, and any fragment thereof.

18. The kit of claim 16, wherein the probes are attached to a solid support.

19. The kit of claim 18, wherein the probes are attached to microparticles.

20. The kit of claim 18, wherein the probes are attached to an array.

21. The kit of claim 19 further comprising reagents for performing a Luminex assay.

22. A CYP2D6 amplification product generated by a PCR reaction containing human genomic DNA and at least one primer pair as set forth in claim 6.

23. A collection of CYP2D6-related amplification products, wherein said collection comprises at least two amplification products generated by a PCR reaction, said PCR reaction containing human genomic DNA and at least two primer pairs as set forth in claim 6.

24. The collection of claim 23, wherein said human genomic DNA comprises a CYP2D6 allele selected from the group consisting of CYP2D6*2A, CYP2D6*12, CYP2D6*4, CYP2D6*10, CYP2D6*11, CYP2D6*17, CYP2D6*6, CYP2D6*8, CYP2D6*3, CYP2D6*9, CYP2D6*2, CYP2D6*7, CYP2D6*5 (gene deletion), and CYP2D6 gene duplication.

25. A collection of CYP2D6-related amplification products, said amplification products comprising at least one amplification product generated by a PCR reaction, said PCR reaction containing human genomic DNA and the primer pairs of the kit of claim 15.

26. The collection of claim 25, wherein said human genomic DNA comprises a CYP2D6 allele selected from the group consisting of CYP2D6*2A, CYP2D6*12, CYP2D6*10, CYP2D6*11, CYP2D6*17, CYP2D6*6, CYP2D6*8, CYP2D6*4, CYP2D6*3, CYP2D6*9, CYP2D6*2, CYP2D6*7, CYP2D6*5 (gene deletion), and CYP2D6 gene duplication.

27. A method for determining which of a plurality of CYP2D6 polymorphic variants is present in an individual, the method comprising steps of:
(a) contacting a sample containing nucleic acid obtained from the individual with at least one allele-specific extension probe, wherein said extension probe is complementary to a target sequence of CYP2D6 immediately adjacent to a polymorphic position and terminates at its 3′ end at a polymorphic position in the CYP2D6 sequence, so that the probe hybridizes to a polymorphic variant that contains a nucleotide complementary to the 3′ terminal nucleotide of the probe to form a hybrid;
(b) subjecting the hybrid formed to conditions suitable for primer extension to form an extension product; and
(c) detecting any extension product, wherein detection of an extension product is indicative of the presence of one particular polymorphic variant at the CYP2D6 polymorphic position.

28. The method of claim 27, wherein the polymorphic position is selected from the group consisting of: −1584 C>G, 100 C>T, 124 G>A, 833 G>C, 1023 C>T, 1707 T>del, 1758 G>T, 1846 G>A, 2549 A>del, 2613-2615 del AGA, 2850 C>T, and 2935 A>C.

29. The method of claim 28, wherein said extension probe comprises a sequence selected from the group consisting of SEQ ID NOs. 11-83, and any active fragment thereof.

30. The method of claim 27, wherein the step of contacting comprises contacting the nucleic acid with a plurality of allele-specific probes, said plurality of allele-specific extension probes comprising at least one pair of extension probes comprising a first extension probe comprising a 3′ portion that is complementary to a CYP2D6 target sequence immediately adjacent to a polymorphic position and that has a 3′-terminal nucleotide that is complementary to a non-mutated/wild-type base at said polymorphic position, and a second extension probe comprising a 3′ portion that is complementary to CYP2D6 target sequence immediately adjacent to the polymorphic position and that has a 3′-terminal nucleotide that is complementary to a mutated/mutant base at said polymorphic position.

31. The method of claim 27, wherein said sample comprises DNA obtained by amplification.

32. The method of claim 31, wherein said amplification is performed using a plurality of primers comprising sequences selected from the group consisting of SEQ ID NOs. 1-10, and any active fragments thereof.

33. The method of claim 27, wherein the detecting step comprises determining which of at least two polymorphic variants exists at a polymorphic site.

34. The method of claim 27, further comprising a step of selecting a therapeutic regimen for the individual, wherein the therapeutic regimen is selected at least in part on the basis of the presence of one or more of the plurality of CYP2D6 polymorphic variants in the individual.


SEQUENCE LISTING (88 sequences)

SEQ ID NO: 1 - DNA, 20 bp, Artificial Sequence
  Amplification primer sequences
cgccgtcaagctttccgaca

SEQ ID NO: 2 - DNA, 20 bp, Artificial Sequence
  Amplification primer sequences
cgccctcgtccccatgctca

SEQ ID NO: 3 - DNA, 21 bp, Artificial Sequence
  Amplification primer sequences
cagctggaatccggtgtcgaa

SEQ ID NO: 4 - DNA, 21 bp, Artificial Sequence
  Amplification primer sequences
cggccctgacactccttcttg

SEQ ID NO: 5 - DNA, 18 bp, Artificial Sequence
  Amplification primer sequences
gcagggagcccaccgtag

SEQ ID NO: 6 - DNA, 20 bp, Artificial Sequence
  Amplification primer sequences
cgcctctccctccatacctc

SEQ ID NO: 7 - DNA, 22 bp, Artificial Sequence
  Amplification primer sequences
tacaggcatgagctaaggcacc

SEQ ID NO: 8 - DNA, 23 bp, Artificial Sequence
  Amplification primer sequences
ctgggattacaggcatgagctaa

SEQ ID NO: 9 - DNA, 21 bp, Artificial Sequence
  Probe sequences
acggaagacaaatcatggcgt

SEQ ID NO: 10 - DNA, 21 bp, Artificial Sequence
  Amplification primer sequences
tcaactttcccttagccgtca

SEQ ID NO: 11 - DNA, 22 bp, Artificial Sequence
  Probe sequences
cctggacaacttggaagaaccc

SEQ ID NO: 12 - DNA, 22 bp, Artificial Sequence
  Probe sequences
cctggacaacttggaagaaccg

SEQ ID NO: 13 - DNA, 18 bp, Artificial Sequence
  Probe sequences
gctgggctgcacgctacc

SEQ ID NO: 14 - DNA, 18 bp, Artificial Sequence
  Probe sequences
gctgggctgcacgctact

SEQ ID NO: 15 - DNA, 19 bp, Artificial Sequence
  Probe sequences
cgctgggctgcacgctacc

SEQ ID NO: 16 - DNA, 19 bp, Artificial Sequence
  Probe sequences
cgctgggctgcacgctact

SEQ ID NO: 17 - DNA, 17 bp, Artificial Sequence
  Probe sequences
ctgggctgcacgctacc

SEQ ID NO: 18 - DNA, 17 bp, Artificial Sequence
  Probe sequences
ctgggctgcacgctact

SEQ ID NO: 19 - DNA, 15 bp, Artificial Sequence
  Probe sequences
cagggggcctggtgg

SEQ ID NO: 20 - DNA, 16 bp, Artificial Sequence
  Probe sequences
gcagggggcctggtga

SEQ ID NO: 21 - DNA, 16 bp, Artificial Sequence
  Probe sequences
gcagggggcctggtgg

SEQ ID NO: 22 - DNA, 17 bp, Artificial Sequence
  Probe sequences
ggcagggggcctggtga

SEQ ID NO: 23 - DNA, 14 bp, Artificial Sequence
  Probe sequences
agggggcctggtgg

SEQ ID NO: 24 - DNA, 15 bp, Artificial Sequence
  Probe sequences
cagggggcctggtga

SEQ ID NO: 25 - DNA, 15 bp, Artificial Sequence
  Probe sequences
ccctgccactgcccg

SEQ ID NO: 26 - DNA, 16 bp, Artificial Sequence
  Probe sequences
cccctgccactgccca

SEQ ID NO: 27 - DNA, 16 bp, Artificial Sequence
  Probe sequences
cccctgccactgcccg

SEQ ID NO: 28 - DNA, 17 bp, Artificial Sequence
  Probe sequences
ccccctgccactgccca

SEQ ID NO: 29 - DNA, 14 bp, Artificial Sequence
  Probe sequences
cctgccactgcccg

SEQ ID NO: 30 - DNA, 15 bp, Artificial Sequence
  Probe sequences
ccctgccactgccca

SEQ ID NO: 31 - DNA, 17 bp, Artificial Sequence
  Probe sequences
agcaggttgcccagccc

SEQ ID NO: 32 - DNA, 18 bp, Artificial Sequence
  Probe sequences
cagcaggttgcccagcct

SEQ ID NO: 33 - DNA, 19 bp, Artificial Sequence
  Probe sequences
cctgaccctccctctgcag

SEQ ID NO: 34 - DNA, 19 bp, Artificial Sequence
  Probe sequences
cctgaccctccctctgcac

SEQ ID NO: 35 - DNA, 14 bp, Artificial Sequence
  Probe sequences
agcggcgccgcaac

SEQ ID NO: 36 - DNA, 14 bp, Artificial Sequence
  Probe sequences
agcggcgccgcaag

SEQ ID NO: 37 - DNA, 17 bp, Artificial Sequence
  Probe sequences
ccgcctgtgcccatcac

SEQ ID NO: 38 - DNA, 18 bp, Artificial Sequence
  Probe sequences
ccggcctgtgcccatcat

SEQ ID NO: 39 - DNA, 18 bp, Artificial Sequence
  Probe sequences
cccgcctgtgcccatcac

SEQ ID NO: 40 - DNA, 18 bp, Artificial Sequence
  Probe sequences
cccgcctgtgcccatcat

SEQ ID NO: 41 - DNA, 16 bp, Artificial Sequence
  Probe sequences
cgcctgtgcccatcac

SEQ ID NO: 42 - DNA, 16 bp, Artificial Sequence
  Probe sequences
cgcctgtgcccatcat

SEQ ID NO: 43 - DNA, 18 bp, Artificial Sequence
  Probe sequences
cgaaacccaggatctggg

SEQ ID NO: 44 - DNA, 19 bp, Artificial Sequence
  Probe sequences
ccgaaacccaggatctgga

SEQ ID NO: 45 - DNA, 21 bp, Artificial Sequence
  Probe sequences
gcaagaagtcgctggagcagt

SEQ ID NO: 46 - DNA, 20 bp, Artificial Sequence
  Probe sequences
caagaagtcgctggagcagg

SEQ ID NO: 47 - DNA, 22 bp, Artificial Sequence
  Probe sequences
ggcaagaagtcgctggagcagt

SEQ ID NO: 48 - DNA, 21 bp, Artificial Sequence
  Probe sequences
gcaagaagtcgctggagcagg

SEQ ID NO: 49 - DNA, 20 bp, Artificial Sequence
  Probe sequences
caagaagtcgctggagcagt

SEQ ID NO: 50 - DNA, 19 bp, Artificial Sequence
  Probe sequences
aagaagtcgctggagcagg

SEQ ID NO: 51 - DNA, 17 bp, Artificial Sequence
  Probe sequences
gcctcctcggtcaccca

SEQ ID NO: 52 - DNA, 17 bp, Artificial Sequence
  Probe sequences
gcctcctcggtcacccc

SEQ ID NO: 53 - DNA, 18 bp, Artificial Sequence
  Probe sequences
ccttcgccaaccactccg

SEQ ID NO: 54 - DNA, 19 bp, Artificial Sequence
  Probe sequences
gccttcgccaaccactcct

SEQ ID NO: 55 - DNA, 19 bp, Artificial Sequence
  Probe sequences
cttctgcccatcacccacc

SEQ ID NO: 56 - DNA, 19 bp, Artificial Sequence
  Probe sequences
cttctgcccatcacccaca

SEQ ID NO: 57 - DNA, 17 bp, Artificial Sequence
  Probe sequences
gcatctcccacccccag

SEQ ID NO: 58 - DNA, 17 bp, Artificial Sequence
  Probe sequences
gcatctcccacccccaa

SEQ ID NO: 59 - DNA, 18 bp, Artificial Sequence
  Probe sequences
cgcatctcccacccccag

SEQ ID NO: 60 - DNA, 18 bp, Artificial Sequence
  Probe sequences
cgcatctcccacccccaa

SEQ ID NO: 61 - DNA, 16 bp, Artificial Sequence
  Probe sequences
catctcccacccccag

SEQ ID NO: 62 - DNA, 16 bp, Artificial Sequence
  Probe sequences
catctcccacccccaa

SEQ ID NO: 63 - DNA, 16 bp, Artificial Sequence
  Probe sequences
ggcgaaaggggcgtcc

SEQ ID NO: 64 - DNA, 15 bp, Artificial Sequence
  Probe sequences
gcgaaaggggcgtcc

SEQ ID NO: 65 - DNA, 16 bp, Artificial Sequence
  Probe sequences
ggcgaaaggggcgtct

SEQ ID NO: 66 - DNA, 23 bp, Artificial Sequence
  Probe sequences
ggatgagctgctaactgagcaca

SEQ ID NO: 67 - DNA, 22 bp, Artificial Sequence
  Probe sequences
gatgagctgctaactgagcacg

SEQ ID NO: 68 - DNA, 21 bp, Artificial Sequence
  Probe sequences
ccttcctggcagagatggaga

SEQ ID NO: 69 - DNA, 20 bp, Artificial Sequence
  Probe sequences
cttcctggcagagatggagg

SEQ ID NO: 70 - DNA, 22 bp, Artificial Sequence
  Probe sequences
gccttcctggcagagatggaga

SEQ ID NO: 71 - DNA, 21 bp, Artificial Sequence
  Probe sequences
cttcctggcagagatggaggt

SEQ ID NO: 72 - DNA, 22 bp, Artificial Sequence
  Probe sequences
ccttcctggcagagatggaggt

SEQ ID NO: 73 - DNA, 21 bp, Artificial Sequence
  Probe sequences
ccttcctggcagagatggagg

SEQ ID NO: 74 - DNA, 20 bp, Artificial Sequence
  Probe sequences
cttcctggcagagatggaga

SEQ ID NO: 75 - DNA, 20 bp, Artificial Sequence
  Probe sequences
cttcctggcagagatggagg

SEQ ID NO: 76 - DNA, 23 bp, Artificial Sequence
  Probe sequences
gcagcttcatgatgagaacctgc

SEQ ID NO: 77 - DNA, 25 bp, Artificial Sequence
  Probe sequences
agcagcttcaatgatgagaacctgt

SEQ ID NO: 78 - DNA, 23 bp, Artificial Sequence
  Probe sequences
cagcttcaatgatgagaacctgc

SEQ ID NO: 79 - DNA, 24 bp, Artificial Sequence
  Probe sequences
gcagcttcaatgatgagaacctgt

SEQ ID NO: 80 - DNA, 22 bp, Artificial Sequence
  Probe sequences
agcttcaatgatgagaacctgc

SEQ ID NO: 81 - DNA, 22 bp, Artificial Sequence
  Probe sequences
agcttcaatgatgagaacctgt

SEQ ID NO: 82 - DNA, 22 bp, Artificial Sequence
  Probe sequences
gcctcctgctcatgatcctaca

SEQ ID NO: 83 - DNA, 22 bp, Artificial Sequence
  Probe sequences
gcctcctgctcatgatcctacc

SEQ ID NO: 84 - DNA, 17 bp, Artificial Sequence
  Probe sequences
ggaggcgctgagagcga

SEQ ID NO: 85 - DNA, 18 bp, Artificial Sequence
  Probe sequences
ccatacctccccgcaagc

SEQ ID NO: 86 - DNA, 21 bp, Artificial Sequence
  Probe sequences
cctcagggatgctgctgtaca

SEQ ID NO: 87 - DNA, 20 bp, Artificial Sequence
  Probe sequences
gcagtgagccgagatcacac

SEQ ID NO: 88 - DNA, 25 bp, Artificial Sequence
  Probe sequences
tgcactccagtctgggtgataagta